(19) United States
(12) Patent Application Publication
(10) Pub. No.: US 2021/0212950 A1
(43) Pub. Date: Jul. 15, 2021

(54) ORALLY DELIVERABLE FORMULATION TO PREVENT ALL CAUSE MORTALITY AND CARDIOVASCULAR EVENTS

(71) Applicant: Resurgent Pharmaceuticals, Inc., Minneapolis, MN (US)

(72) Inventor: Eric Greenbaum, Minneapolis, MN (US)

(21) Appl. No.: 17/150,361

(22) Filed: Jan. 15, 2021

Related U.S. Application Data
(60) Provisional application No. 62/961,496, filed on Jan. 15, 2020.

(51) Int. Cl. A61K 9/20; A61K 36/67; A61K 9/00; A61K 31/05; A61K 31/352

(52) U.S. Cl. CPC A61K 9/2018; A61K 36/67; A61K 9/006; A61K 31/352; A61K 9/205; A61K 31/05; A61K 9/0056

(57) ABSTRACT

The present disclosure describes materials and methods for providing the health benefits associated with chili peppers and cannabis to subjects in need thereof. For example, an oral formulation including at least one tissue from a fruit of the Capsicum genus, an extract therefrom, or a combination thereof, at least one cannabinoid, and a pharmaceutically acceptable excipient is provided. The oral formulation can be an oral spray, compressed tablet, or a candy-style lozenge.

### FIELD OF THE INVENTION

The invention relates generally to combination products that include cannabis or extracts therefrom with chili peppers or extracts therefrom.

### BACKGROUND OF THE INVENTION

Diets rich in chili peppers (fruit of Capsicum plants) have been shown to offer material health benefits. In particular, a diet rich with chili peppers has been associated with a reduction in all-cause mortality and cardiovascular events (Spence, J Am Coll Cardiol. 2019 December, 74 (25) 3150-3152; Bonaccio et al., J Am Coll Cardiol. 2019 December, 74 (25) 3139-3149). The health advantages associated with chili peppers are frequently ascribed to the high content of capsaicin, which is far more abundant in chili than in sweet non-spicy peppers. Cannabis and extracts therefrom have also been shown to confer meaningful health benefits. Thus, there is a need in the art of nutritional supplements and pharmacotherapy to develop new products using both chili peppers and cannabis for easy administration of these protective and beneficial compounds.

### SUMMARY

The present disclosure describes materials and methods for providing the health benefits associated with chili peppers and cannabis to subjects in need thereof.

In a first aspect, the present disclosure provides an oral formulation including at least one at least one tissue from a fruit of a species of Capsicum, an extract therefrom, or a combination thereof; at least one cannabinoid; and a pharmaceutically acceptable excipient. The tissue can be dried and ground. The oral formulation can include about 10 mg to 1000 mg tissue or about 100 mg to 500 mg tissue. The oral formulation can include a quantity of tissue sufficient to achieve a predetermined amount of polyphenol, flavonoid, or capsaicin in the formulation. The tissue and extract can lack or substantially lack capsaicin. The tissue is selected from a cultivar of the Capsicum species with a Scoville heat unit (SHU) value of less than 50,000. The oral formulation can be an oral spray. The oral formulation can be a compressed tablet. The oral formulation can be a candy-style lozenge. The oral formulation can further contain at least one sugar alcohol. The oral formulation can further contain a hydrocolloid selected from the group consisting of gelatin, gum acacia, carob gum, carrageenan, ghatti gum, guar gum, karaya gum, pectin, tragacanth gum, locust bean gum, xanthan gum and combinations thereof. The pharmaceutically acceptable excipient can be selected from the group consisting of emulsifiers, surfactants, colorants, flavorants, taste modifiers, buffering agents, pH adjusting agents, stabilizers, sweeteners and combinations thereof. The at least one cannabinoid can be a water-soluble powder including a cannabinoid oil, isomalt, a hydrocolloid, and an emulsifier. The hydrocolloid can be gum acacia (AKA gum Arabic) and the emulsifier can be quillaja extract.

In a second aspect, the present disclosure provides a method for improving health of a subject in need thereof comprising administering to the subject an oral formulation comprising: at least one tissue from a fruit of a species of Capsicum, an extract therefrom, or a combination thereof; at least one cannabinoid; and a pharmaceutically acceptable excipient. The oral formulation can be administered at least once a week for four weeks. The oral formulation can be administered at least three times a week.

In a third aspect, the present disclosure provides a method for reducing the probability of a cardiac event in a patient in need thereof comprising administering to the patient an oral formulation comprising: at least one tissue from a fruit of a species of Capsicum, an extract therefrom, or a combination thereof; at least one cannabinoid; and a pharmaceutically acceptable excipient.

### DETAILED DESCRIPTION

These and other systems, methods, objects, features, and advantages of the present disclosure will be apparent to those skilled in the art from the following detailed description of the embodiments and drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from context.

As used herein, “all-cause mortality” refers to the lowering effect of the intervention on the risk of death occurring in a population, regardless of the cause, as compared to estimates for all-cause mortality adjusted for one or more of age, sex, educational level, occupational class, smoking, leisure-time physical activity, cardiovascular disease, cancer, drugs for diabetes, lipid-lowering drugs, medication for hypertension, diet and energy intake (kcal/day).

The term “candy-style lozenge” as used herein refers to a solid preparation that is intended to dissolve or disintegrate slowly in the mouth, keeping the active ingredients in contact with the oral cavity for an extended period of time. A candy-style lozenge can be made by compression or molding, include one or more of flavorings, colorings, and sweeteners. A candy-style lozenge can be hard, soft or chewable, and include forms such as lozenges include lollipops and gummies.

The term “cardiac event” as used herein refers to any severe or acute cardiovascular condition, including, but not limited to cardiac arrest, myocardial infarction, coronary artery disease, ECG evidence of past myocardial infarction or newly recognized left bundle branch block, angina, coronary artery bypass grafting, chest pain without significant coronary artery disease, obstructive coronary stenosis, coronary artery spasm, myocardial bridge, coronary angioplasty and exertion-induced cardiac events.

The term “tissue” as used herein refers to one or more plant tissues or organs which are obtained from a whole plant or plant parts, preferably a cannabis or capsicum plant. Plant parts include vegetative structures (for example, leaves, stems), roots, floral organs/structures, seed (including embryo, endosperm, and seed coat), plant tissue (for example, vascular tissue, ground tissue, and the like) and cells. The plant part can be fresh or dried (e.g., powdered).

The term “extract” as used herein with respect to Capsicum fruit refers to water, organic solvent (e.g., hexane), and oil extracts of ripe or unripe fruit or powders thereof. The extracted composition can be spray dried to provide a Capsicum fruit extract as a powder. The extract includes at least one of the endogenous phytocompounds present in the source tissue, such as capsaicin, dihydrocapsaicin, nordihydrocapsaicin, omocapsaicins I and II, octyl-, nonyl-, and decylvanillamide, capsanthin, kryptxanthin, zeaxanthin, capsorubin, lutein; α-, β-, and neo-o-carotene, β-carotene epoxide, ascorbic acid, provitamins A, B1, B2, C, E, and PP, nicotinic acid amide, tocopherols, cynaroside, quercetin, apiin, luteolin-7-monoglucoside, capsicoside, citric and malonic acid, 2-methoxy-3-isobutylpyrazine, citrozanthin, violaxanthin, and capsorubin.

### A. Cannabinoids

The cannabinoids are a class of molecules primarily obtained through the extraction of cannabis plant material, although synthetic and/or bioreactor production, or other methods may also be used to obtain or modify them. Cannabinoids include, but are not limited to: THC (tetrahydrocannabinol); THCA (tetrahydrocannabinolic acid); CBD (cannabidiol); CBDA (cannabidiolic acid); CBN (cannabinol); CBG (cannabigerol); CBC (cannabichromene); CBL (cannabicyclol); CBV (cannabivarin); THCV (tetrahydrocannabivarin); CBDV (cannabidivarin); CBCV (cannabichromevarin); CBGV (cannabigerovarin); CBGM (cannabigerol monomethyl ether); CBE (cannabielsoin); and CBT (cannabicitran). Naturally occurring cannabinoids may be characterized by their type. Such a characterization includes, but is not limited to: Cannabigerol-type (CBG-type); Cannabichromene-type (CBC-type); Cannabidiol-type (CBD-type); Cannabinodiol-type (CBND-type); Tetrahydrocannabinol-type (THC-type); Cannabinol-type (CBN-type); Cannabitriol-type (CBT-type); Cannabielsoin-type (CBE-type); the Isocannabinoids; Cannabicyclol-type (CBL-type); Cannabicitran-type (CBT-type) and Cannabichromanone-type (CBCN-type). Chemical structures for exemplary naturally occurring cannabinoids are provided in TABLE 1.

TABLE 1
Non-exhaustive list of plant cannabinoids by type.
Cannabigerol-type (CBG)

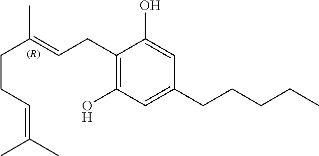

Cannabigerol
(E)-CBG-C₅

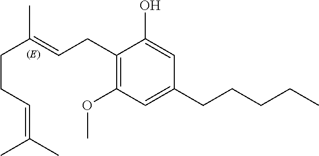

Cannabigerol
monomethyl ether
(E)-CBGM-C₅ A

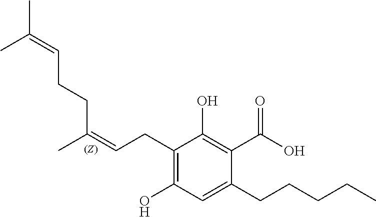

Cannabinerolic acid A
(Z)-CBGA-C₅ A

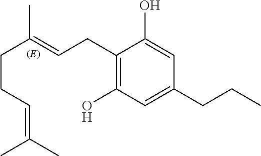

Cannabigerovarin
(E)-CBGV-C₃

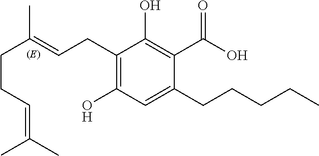

Cannabigerolic acid A
(E)-CBGA-C₅ A

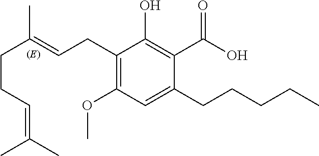

Cannabigerolic acid A
monomethyl ether
(E)-CBGAM-C₅ A

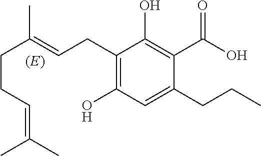 [SMILES: CCCc1cc(O)c(C/C=C(\C)CCC=C(C)C)c(O)c1C(=O)O]

Cannabigerovarinic acid A
(E)-CBGVA-C₃ A
Cannabichromene-type (CBC)

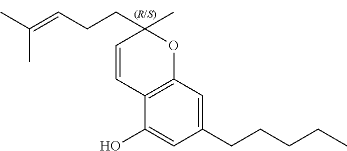 [SMILES: CCCCCc1cc(O)c2c(c1)OC(C)(CCC=C(C)C)C=C2]

(±)-Cannabichromene
CBC-C₅

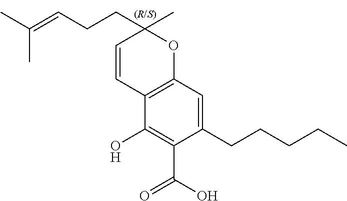 [SMILES: CCCCCc1cc2c(c(O)c1C(=O)O)C=CC(C)(CCC=C(C)C)O2]

(±)-Cannabichromenic acid A
CBCA-C₅ A

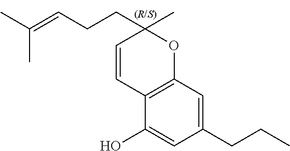 [SMILES: CCCc1cc(O)c2c(c1)OC(C)(CCC=C(C)C)C=C2]

(±)-Cannabivarichromene,
(±)-Cannabichromevarin
CBCV-C₃

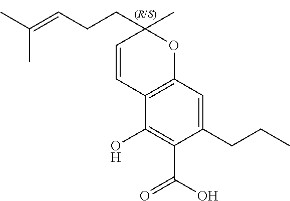 [SMILES: CCCc1cc2c(c(O)c1C(=O)O)C=CC(C)(CCC=C(C)C)O2]

(±)-Cannabichromevarinic acid A
CBCVA-C₃ A
Cannabidiol-type (CBD)

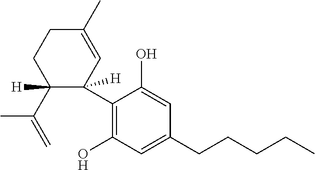 [SMILES: C=C(C)[C@@H]1CCC(C)=C[C@H]1c1c(O)cc(CCCCC)cc1O]

(−)-Cannabidiol
CBD-C₅

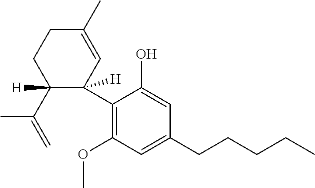 [SMILES: C=C(C)[C@@H]1CCC(C)=C[C@H]1c1c(O)cc(CCCCC)cc1OC]

Cannabidiol momomethyl ether
CBDM-C₅

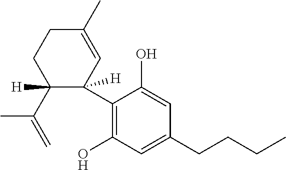 [SMILES: C=C(C)[C@@H]1CCC(C)=C[C@H]1c1c(O)cc(CCCC)cc1O]

Cannabidiol-C₄
CBD-C₄

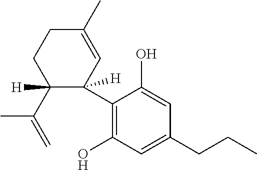 [SMILES: C=C(C)[C@@H]1CCC(C)=C[C@H]1c1c(O)cc(CCC)cc1O]

(−)-Cannabidivarin
CBDV-C₃

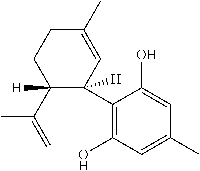 [SMILES: C=C(C)[C@@H]1CCC(C)=C[C@H]1c1c(O)cc(C)cc1O]

Cannabidiorcol
CBD-C₁

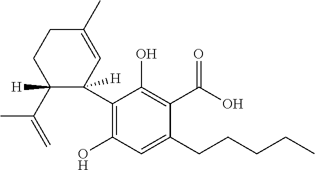 [SMILES: C=C(C)[C@@H]1CCC(C)=C[C@H]1c1c(O)cc(CCCCC)c(C(=O)O)c1O]

Cannabidiolic acid
CBDA-C₅

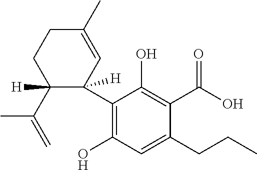 [SMILES: C=C(C)[C@@H]1CCC(C)=C[C@H]1c1c(O)cc(CCC)c(C(=O)O)c1O]

Cannabidivarinic acid
CBDVA-C₃
Cannabinodiol-type (CBND)

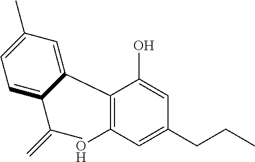

Cannabinodiol
CBND-C₅

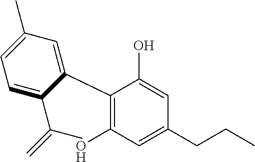

Cannabinodivarin
CBND-C₃
Tetrahydrocannabinol-type (THC)

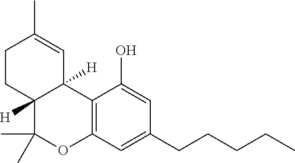 [SMILES: CCCCCc1cc(O)c2c(c1)OC(C)(C)[C@@H]1CCC(C)=C[C@@H]21]

Δ⁹-Tetrahydrocannabinol
Δ⁹-THC-C₅

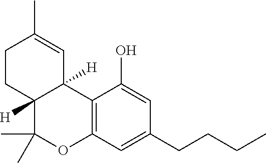 [SMILES: CCCCc1cc(O)c2c(c1)OC(C)(C)[C@@H]1CCC(C)=C[C@@H]21]

Δ⁹-Tetrahydrocannabinol-C₄
Δ⁹-THC-C₄

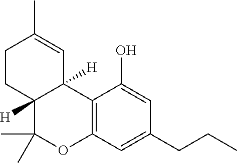 [SMILES: CCCc1cc(O)c2c(c1)OC(C)(C)[C@@H]1CCC(C)=C[C@@H]21]

Δ⁹-Tetrahydrocannabivarin
Δ⁹-THCV-C₃

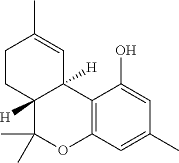

Δ⁹-Tetrahydrocannabiorcol
Δ⁹-THCO-C₁

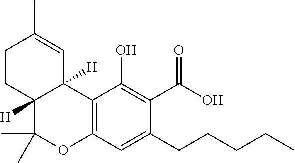 [SMILES: CCCCCc1cc2c(c(O)c1C(=O)O)[C@@H]1C=C(C)CC[C@H]1C(C)(C)O2]

Δ⁹-Tetrahydrocannabinolic acid A
Δ⁹-THCA-C₅ A

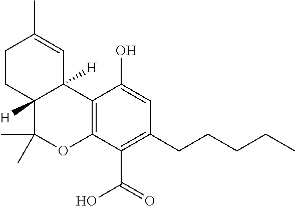 [SMILES: CCCCCc1cc(O)c2c(c1C(=O)O)OC(C)(C)[C@@H]1CCC(C)=C[C@@H]21]

Δ⁹-Tetrahydrocannabinolic acid B
Δ⁹-THCA-C₅ B

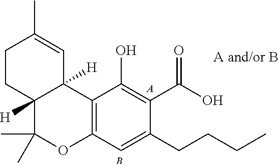

Δ⁹-Tetrahydrocannabinolic acid-C₄
A and/or B
Δ⁹-THCA-C₄ A and/or B

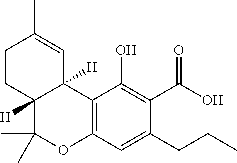 [SMILES: CCCc1cc2c(c(O)c1C(=O)O)[C@@H]1C=C(C)CC[C@H]1C(C)(C)O2]

Δ⁹-Tetrahydrocannabivarinic acid A
Δ⁹-THCVA-C₃ A

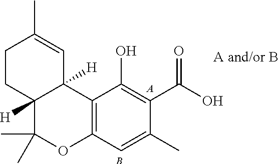

Δ⁹-Tetrahydrocannabiorcolic acid
A and/or B
Δ⁹-THCOA-C₁ A and/or B

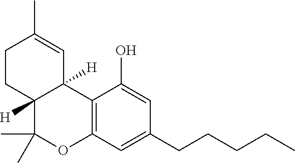 [SMILES: CCCCCc1cc(O)c2c(c1)OC(C)(C)[C@@H]1CCC(C)=C[C@@H]21]

(−)-Δ⁸-trans-(6aR,10aR)-
Δ⁸-Tetrahydrocannabinol
Δ⁸-THC-C₅

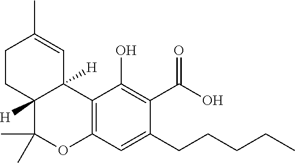 [SMILES: CCCCCc1cc2c(c(O)c1C(=O)O)[C@@H]1C=C(C)CC[C@H]1C(C)(C)O2]

(−)-Δ⁸-trans-(6aR,10aR)-
Tetrahydrocannabinolic acid A
Δ⁸-THCA-C₅ A

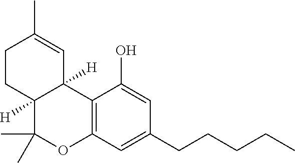 [SMILES: CCCCCc1cc(O)c2c(c1)OC(C)(C)[C@H]1CCC(C)=C[C@@H]21]

(−)-(6as,10aR)-Δ⁹-
Tetrahydrocannabinol
(−)-cis-Δ⁹-THC-C₅
Cannabinol-type (CBN)

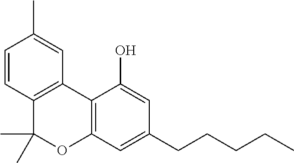 [SMILES: CCCCCc1cc(O)c2c(c1)OC(C)(C)c1ccc(C)cc1-2]

Cannabinol
CBN-C₅

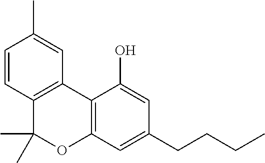 [SMILES: CCCCc1cc(O)c2c(c1)OC(C)(C)c1ccc(C)cc1-2]

Cannabinol-C₄
CBN-C₄

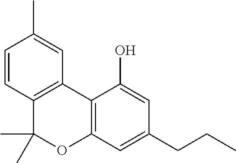 [SMILES: CCCc1cc(O)c2c(c1)OC(C)(C)c1ccc(C)cc1-2]

Cannabivarin
CBN-C₃

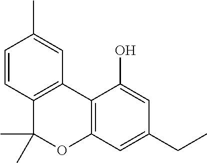 [SMILES: CCc1cc(O)c2c(c1)OC(C)(C)c1ccc(C)cc1-2]

Cannabinol-C₂
CBN-C₂

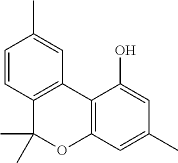

Cannabiorcol
CBN-C₁

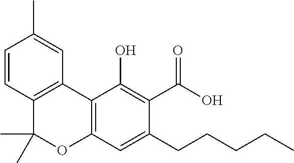 [SMILES: CCCCCc1cc2c(c(O)c1C(=O)O)-c1cc(C)ccc1C(C)(C)O2]

Cannabinolic acid A
CBNA-C₅ A

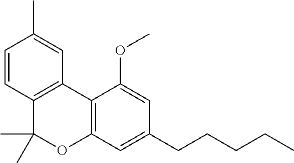 [SMILES: CCCCCc1cc(OC)c2c(c1)OC(C)(C)c1ccc(C)cc1-2]

Cannabinol methyl ether
CBNM-C₅
Cannabitriol-type (CBT)

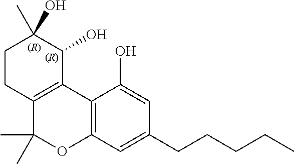

(−)-(9R,10R)-trans-Cannabitriol
(−)-trans-CBT-C₅

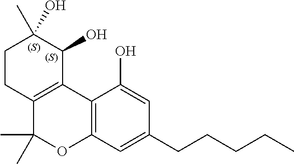

(+)-(9S,10S)-Cannabitriol
(+)-trans-CBT-C₅

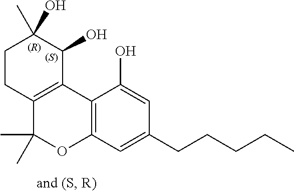

(±)-(9R,10S/9S,10R)-Cannabitriol
(±)-cis-CBT-C₅

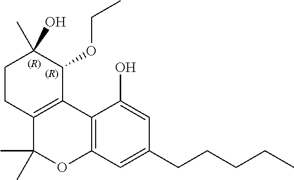

(−)-(9R,10R)-trans-
10-O-Ethyl-cannabitriol
(−)-trans-CBT-OEt-C₅

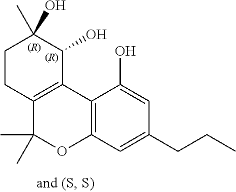

(±)-(9R,10R/9S,10S)-
Cannabitriol-C₃
(±)-trans-CBT-C₃

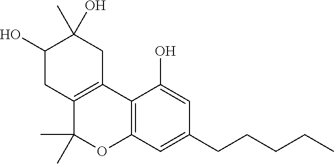 [SMILES: CCCCCc1cc(O)c2c(c1)OC(C)(C)C1=C2CC(C)(O)C(O)C1]

8,9-Dihydroxy-
Δ^{6a(10a)}-tetrahydrocannabinol
8,9-Di-OH-CBT-C₅

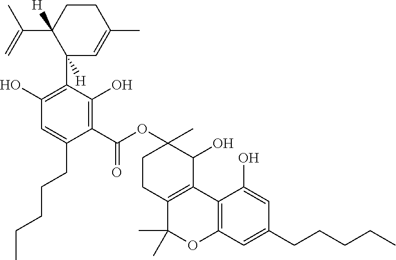 [SMILES: C=C(C)[C@@H]1CCC(C)=C[C@H]1c1c(O)cc(CCCCC)c(C(=O)OC2(C)CCC3=C(c4c(O)cc(CCCCC)cc4OC3(C)C)C2O)c1O]

Cannabidolic acid A
cannabitriol ester
CBDA-C₅ 9-OH-CBT-C₅ ester

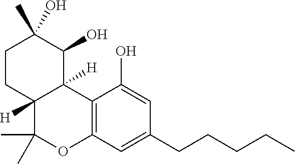 [SMILES: CCCCCc1cc(O)c2c(c1)OC(C)(C)[C@@H]1CC[C@](C)(O)[C@@H](O)[C@@H]21]

(−)-(6aR,9S,10S,10aR)-
9,10-Dihydroxy-hexahydro-
cannabinol, Cannabiripsol
Cannabiripsol-C₅

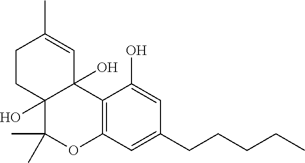 [SMILES: CCCCCc1cc(O)c2c(c1)OC(C)(C)C1(O)CCC(C)=CC21O]

(−)-6a,7,10a-
Trihydroxy-Δ⁹-
tetrahydrocannabinol
(−)-Cannabitetrol

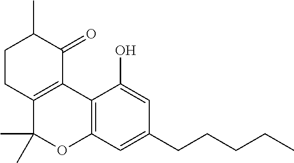 [SMILES: CCCCCc1cc(O)c2c(c1)OC(C)(C)C1=C2C(=O)C(C)CC1]

10-Oxo-Δ^{6a(10a)}-
tetrahydrocannabinol
OTHC
Cannabielsoin-type (CBE)

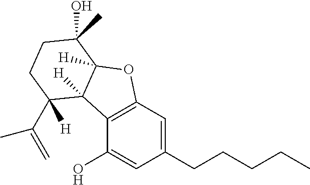

(5aS,6S,9R,9aR)-Cannabielsoin
CBE-C₅

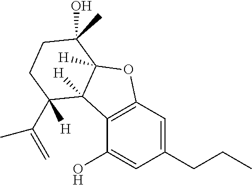

(5aS,6S,9R,9aR)-C₃-Cannabielsoin
CBE-C₃

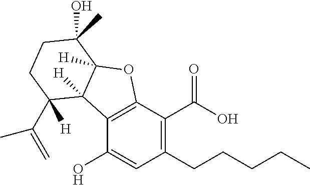

(5aS,6S,9R,9aR)-Cannabielsoic acid A
CBEA-C₅ A

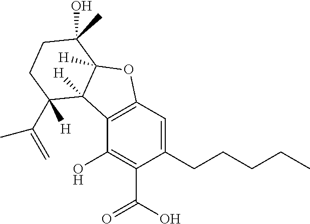

(5aS,6S,9R,9aR)-Cannabielsoic acid B
CBEA-C₅ B

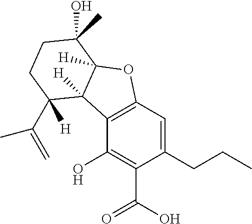

(5aS,6S,9R,9aR)-C₃-Cannabielsoic acid B
CBEA-C₃ B

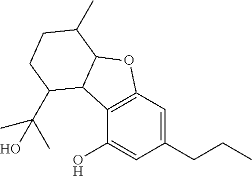 [SMILES: CCCc1cc(O)c2c(c1)OC1C(C)CCC(C(C)(C)O)C21]

Cannabiglendol-C₃
OH-iso-HHCV-C₃

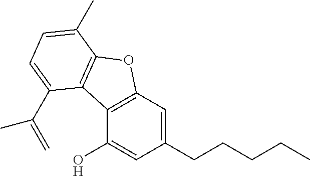 [SMILES: C=C(C)c1ccc(C)c2oc3cc(CCCCC)cc(O)c3c12]

Dehydro-
cannabifuran
DCBF-C₅

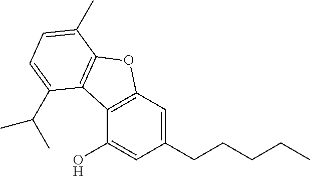 [SMILES: CCCCCc1cc(O)c2c(c1)oc1c(C)ccc(C(C)C)c12]

Cannabifuran
CBF-C₅
Isocannabinoids

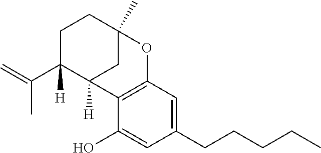 [SMILES: C=C(C)[C@@H]1CC[C@]2(C)C[C@H]1c1c(O)cc(CCCCC)cc1O2]

(−)-Δ⁷-trans-(1R,3R,6R)-
Isotetrahydrocannabinol

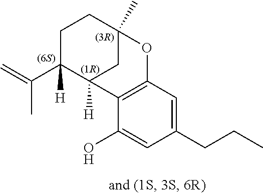 [SMILES: C=C(C)[C@@H]1CC[C@]2(C)C[C@H]1c1c(O)cc(CCC)cc1O2]

(±)-Δ⁷-1,2-cis-(1R,3R,6S/1S,3S,6R)-
Isotetrahydrocannabivarin

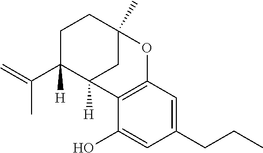 [SMILES: C=C(C)[C@@H]1CC[C@]2(C)C[C@H]1c1c(O)cc(CCC)cc1O2]

(−)-Δ⁷-trans-(1R,3R,6R)-
Isotetrahydrocannabivarin
Cannabicyclol-type (CBL)

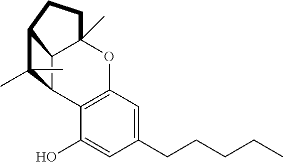

(±)-(1aS,3aR,8bR,8cR)-
Cannabicyclol
CBL-C₅

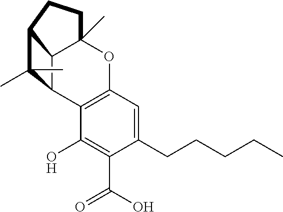

(±)-(1aS,3aR,8bR,8cR)-
Cannabicyclolic acid A
CBLA-C₅ A

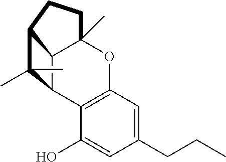

(±)-(1aS,3aR,8bR,8cR)-
Cannabicyclovarin
CBLV-C₃
Cannabicitran-type (CBT)

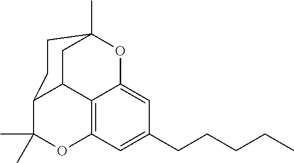 [SMILES: CCCCCc1cc2c3c(c1)OC(C)(C)C1CCC(C)(CC31)O2]

Cannabicitran
CBT-C₅
Cannabichromanone-type (CBCN)

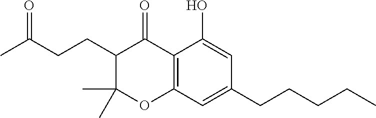 [SMILES: CCCCCc1cc(O)c2c(c1)OC(C)(C)C(CCC(C)=O)C2=O]

Cannabichromanone
CBCN-C₅

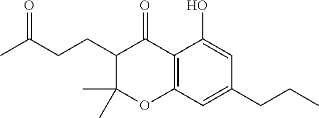 [SMILES: CCCc1cc(O)c2c(c1)OC(C)(C)C(CCC(C)=O)C2=O]

Cannabichromanone-C₃
CBCN-C₃

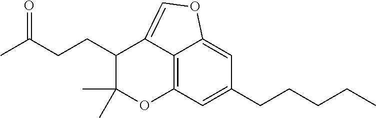 [SMILES: CCCCCc1cc2c3c(coc3c1)C(CCC(C)=O)C(C)(C)O2]

Cannabicoumaronone
CBCON-C₅

In addition to the naturally occurring cannabinoids, a virtually unlimited number of synthetic cannabinoids, with cannabinomimetic properties, exist and are subject to additional creation and or modification. Some of the known synthetic cannabinoids include, but are not limited to: rimonabant, JWH-018, JWH-073, CP-55940; dimethylheptyran; HU-210; HU-331, SR144528, WIN 55,212-2, JWH-133, Levonantradol (Nantrodolum), AM-2201, etc.

### B. Cannabinoid Formulations

In some embodiments the cannabinoids and/or combinations thereof may be processed to improve hydrophilicity using any methods known to those having skill in the art, including but not limited to the creation of emulsions, micro emulsions, nanoemulsions, encapsulation, complexation with solubility enhancers such as, for example, with a cyclodextrin, including but not limited to beta cyclodextrins, sulfobutylether beta cyclodextrins and the like; and complexation with sugar alcohols such as, for example erythritol, isomalt and the like.

Various cannabinoids, used alone or in combination have shown a variety of significant biological effects including but not limited to pain relief, anti-cancer, anti-inflammatory, anti-emetic, anti-convulsant, and many others, including recreational effects.

The formulations described herein can be delivered to a patient/subject, in need of any one of these biological effects using any form suitable for cannabinoid delivery via a desired route of administration. Examples of suitable forms include but are not limited to the products listed in TABLE 2.

TABLE 2
Exemplary dosage forms approved by the US Food and Drug Administration
with the National Cancer Institute Thesaurus code.

| Structured Product Labeling Acceptable Term | FDA NCI Code |
|---|---|
| AEROSOL | C42887 |
| AEROSOL, FOAM | C42888 |
| AEROSOL, METERED | C42960 |
| AEROSOL, POWDER | C42971 |
| AEROSOL, SPRAY | C42889 |
| BAR, CHEWABLE | C42892 |
| BEAD | C42890 |
| CAPSULE | C25158 |
| CAPSULE, COATED | C42895 |
| CAPSULE, COATED PELLETS | C42896 |
| CAPSULE, COATED, EXTENDED RELEASE | C42917 |
| CAPSULE, DELAYED RELEASE | C42902 |
| CAPSULE, DELAYED RELEASE PELLETS | C42904 |
| CAPSULE, EXTENDED RELEASE | C42916 |
| CAPSULE, FILM COATED, EXTENDED RELEASE | C42928 |
| CAPSULE, GELATIN COATED | C42936 |
| CAPSULE, LIQUID FILLED | C42954 |
| CELLULAR SHEET | C100103 |
| CHEWABLE GEL | C134876 |
| CLOTH | C60884 |
| CONCENTRATE | C60891 |
| CREAM | C28944 |
| CREAM, AUGMENTED | C60897 |
| CRYSTAL | C42901 |
| DISC | C43525 |
| DOUCHE | C42679 |
| DRESSING | C42763 |
| ELIXIR | C42912 |
| EMULSION | C42913 |
| ENEMA | C42915 |
| EXTRACT | C42929 |
| FIBER, EXTENDED RELEASE | C60926 |
| FILM | C42932 |
| FILM, EXTENDED RELEASE | C42920 |
| FILM, SOLUBLE | C42984 |
| FOR SOLUTION | C60927 |
| FOR SUSPENSION | C60928 |
| FOR SUSPENSION, EXTENDED RELEASE | C60929 |
| GAS | C42933 |
| GEL | C42934 |
| GEL, DENTIFRICE | C42906 |
| GEL, METERED | C60930 |
| GLOBULE | C42937 |
| GRANULE | C42938 |
| GRANULE, DELAYED RELEASE | C42903 |
| GRANULE, EFFERVESCENT | C42909 |
| GRANULE, FOR SOLUTION | C42939 |
| GRANULE, FOR SUSPENSION | C42940 |
| GRANULE, FOR SUSPENSION, EXTENDED RELEASE | C42921 |
| GUM, CHEWING | C42894 |
| IMPLANT | C42942 |
| INHALANT | C42944 |
| INJECTABLE FOAM | C113106 |
| INJECTABLE, LIPOSOMAL | C60931 |
| INJECTION | C42946 |
| INJECTION, EMULSION | C42914 |
| INJECTION, LIPID COMPLEX | C42950 |
| INJECTION, POWDER, FOR SOLUTION | C42974 |
| INJECTION, POWDER, FOR SUSPENSION | C42976 |
| INJECTION, POWDER, FOR SUSPENSION, EXTENDED | C42977 |
| RELEASE | |
| INJECTION, POWDER, LYOPHILIZED, FOR LIPOSOMAL | C42959 |
| SUSPENSION | |
| INJECTION, POWDER, LYOPHILIZED, FOR SOLUTION | C42957 |
| INJECTION, POWDER, LYOPHILIZED, FOR SUSPENSION | C42958 |
| INJECTION, POWDER, LYOPHILIZED, FOR SUSPENSION, | C42956 |
| EXTENDED RELEASE | |
| INJECTION, SOLUTION | C42945 |
| INJECTION, SOLUTION, CONCENTRATE | C42899 |
| INJECTION, SUSPENSION | C42995 |
| INJECTION, SUSPENSION, EXTENDED RELEASE | C42926 |
| INJECTION, SUSPENSION, LIPOSOMAL | C42951 |
| INJECTION, SUSPENSION, SONICATED | C42988 |
| INSERT | C60933 |
| INSERT, EXTENDED RELEASE | C42922 |
| INTRAUTERINE DEVICE | C47915 |
| IRRIGANT | C42947 |
| JELLY | C42948 |
| KIT | C47916 |
| LINIMENT | C42949 |
| LIPSTICK | C42952 |
| LIQUID | C42953 |
| LIQUID, EXTENDED RELEASE | C60934 |
| LOTION | C29167 |
| LOTION, AUGMENTED | C60957 |
| LOTION/SHAMPOO | C60958 |
| LOZENGE | C42955 |
| MOUTHWASH | C29269 |
| NOT APPLICABLE | C48624 |
| OIL | C42965 |
| OINTMENT | C42966 |
| OINTMENT, AUGMENTED | C60984 |
| PASTE | C42967 |
| PASTE, DENTIFRICE | C42907 |
| PASTILLE | C60985 |
| PATCH | C42968 |
| PATCH, EXTENDED RELEASE | C42923 |
| PATCH, EXTENDED RELEASE, ELECTRICALLY | C42911 |
| CONTROLLED | |
| PELLET | C42969 |
| PELLET, IMPLANTABLE | C42943 |
| PELLETS, COATED, EXTENDED RELEASE | C42918 |
| PILL | C25394 |
| PLASTER | C42970 |
| POULTICE | C47913 |
| POWDER | C42972 |
| POWDER, DENTIFRICE | C42908 |
| POWDER, FOR SOLUTION | C42973 |
| POWDER, FOR SUSPENSION | C42975 |
| POWDER, METERED | C42961 |
| RING | C60988 |
| RINSE | C42979 |
| SALVE | C42980 |
| SHAMPOO | C42981 |
| SHAMPOO, SUSPENSION | C42982 |
| SOAP | C42983 |
| SOLUTION | C42986 |
| SOLUTION, CONCENTRATE | C42898 |
| SOLUTION, FOR SLUSH | C42987 |
| SOLUTION, GEL FORMING/DROPS | C60994 |
| SOLUTION, GEL FORMING, EXTENDED RELEASE | C42935 |
| SOLUTION/DROPS | C60992 |
| SPONGE | C47912 |
| SPRAY | C42989 |
| SPRAY, METERED | C42962 |
| SPRAY, SUSPENSION | C42990 |
| STICK | C42991 |
| STRIP | C47914 |
| SUPPOSITORY | C42993 |
| SUPPOSITORY, EXTENDED RELEASE | C42924 |
| SUSPENSION | C42994 |
| SUSPENSION, EXTENDED RELEASE | C42925 |
| SUSPENSION/DROPS | C60995 |
| SWAB | C47898 |
| SYRUP | C42996 |
| SYSTEM | C17423 |
| TABLET | C42998 |
| TABLET, CHEWABLE | C42893 |
| TABLET, CHEWABLE, EXTENDED RELEASE | C124794 |
| TABLET, COATED | C42897 |
| TABLET, COATED PARTICLES | C60997 |
| TABLET, DELAYED RELEASE | C42905 |
| TABLET, DELAYED RELEASE PARTICLES | C42997 |
| TABLET, EFFERVESCENT | C42910 |
| TABLET, EXTENDED RELEASE | C42927 |
| TABLET, FILM COATED | C42931 |
| TABLET, FILM COATED, EXTENDED RELEASE | C42930 |
| TABLET, FOR SOLUTION | C61004 |
| TABLET, FOR SUSPENSION | C61005 |
| TABLET, MULTILAYER | C42964 |
| TABLET, MULTILAYER, EXTENDED RELEASE | C42963 |
| TABLET, ORALLY DISINTEGRATING | C42999 |
| TABLET, ORALLY DISINTEGRATING, DELAYED | C61006 |
| RELEASE | |
| TABLET, SOLUBLE | C42985 |
| TABLET, SUGAR COATED | C42992 |
| TABLET WITH SENSOR | C147579 |
| TAMPON | C47892 |
| TAPE | C47897 |
| TINCTURE | C43000 |
| TROCHE | C43001 |
| WAFER | C43003 |

### C. Chili Pepper

Chili peppers, as they are colloquially known, are fruits of the Capsicum genus. There are approximately fifty thousand Capsicum cultivars grown worldwide. The USDA-ARS GRIN seed collection contains 6,200 Capsicum accessions alone, which include 4,000 Capsicum annuum accessions. The other Capsicum species in the USDA germplasm repository include: C. chinense, C. baccatum, C. frutescens, C. pubescens, C. cardenasii, C. chacoense, C. flexuosum, C. eximium, C. rhomboideum, C. galapagoense, and C. tovarii.

There are five major species of cultivated Capsicum, C. annuum, C. chinense, C. baccatum, C. frutescens, C. pubescens, and within those species are several “taxonomic varieties”. Because of the ability of many of species to cross and generate inter-specific hybrids, albeit with low success, there are also what is referred to as “complexes” within the genus Capsicum of closely related and sexually compatible species. This includes the Capsicum annuum complex, which includes three closely related species, C. annuum, C. frutescens, and C. chinense. The Major species and their taxonomic varieties include: Capsicum annuum, which includes bell peppers, sweet/Italian peppers, serrano, cayenne, paprika and jalapeños; Capsicum baccatum, which includes the South American varieties, such as aji amarillo, aji limon and criolla sella; Capsicum chinense, which includes all of the Habaneros, Scotch Bonnets, Trinidad Scorpions, the Bhut Jolokia and the Carolina Reaper; Capsicum frutescens, which includes the Tabascos and many of the peppers grown in India; sometimes not distinguished as a species separate from C. annuum; and Capsicum pubescens, which includes the Rocoto and Manzano pepper, and are distinctive plants in having violet flowers, black seeds and hairy dark green leaves and grows as a large, multi-stemmed vine up to 5 meters long.

The species and varieties include many economically important cultivars with different shapes, colors, and flavors that are grown for different purposes, such as spices, vegetables, and herbal medicines. See TABLE 3 for a non-limiting list of cultivars identified by their common names. Some confusion has resulted from the legal term “plant variety”, which is used interchangeably with “cultivar” (not with “taxonomic variety”). The terminology around a cultivar also includes terms such as heirloom, open-pollinated, self-pollinating, and hybrid.

Heirloom varieties are typically those that have been selected and grown historically with seed saved every year, and are still maintained today in similar fashion, such as the blocky-type California Wonder. Open-pollinated varieties are those that are maintained without strict barriers to prevent outcrossing and then seed is collected at and stored from each harvest such as the lamuyo-type Marconi Yellow. While open-pollinated varieties are typically true-to-type, there may be occasional outcrossing to other Capsicum varieties that may introduce some heterogeneity.

Hybrid varieties take advantage of a phenomenon called heterosis or hybrid vigor, which occurs in pepper. To generate a hybrid variety, two self-pollinated varieties are intentionally crossed, and all seed from this cross are collected. The new hybrid variety typically is more vigorous than either of the two parents contributing to traits such as higher yield. Hybrid seed if saved will not produce a homogeneous set of plants the next generation, meaning that the two parents will need to be crossed again to generate more hybrid seed. This method is used to produce hybrid Capsicum cultivars such as the blocky-types Double-Up and Orange Blaze. Much of the commercial pepper production uses hybrid varieties for their improved traits.

TABLE 3
Example Capsicum species and cultivars:

| | | | | |
|---|---|---|---|---|
| | | | Scoville Heat | |
| Common Name | Type | Origin | Units (SHU) | Description |

Capsicum Annum

| | | | | |
|---|---|---|---|---|
| Aleppo | | Syria and | 15,000 | Grown in Syria and Turkey |
| | | Turkey | | and used, in coarsely ground, |
| | | | | dried form, as a spice that is |
| | | | | also called Aleppo pepper |
| Anaheim | Anaheim | United States | 500-2,500 | A mild variety of New Mexico |
| | | (US) | | chile. It was later brought |
| | | | | to California from New |
| | | | | Mexico by Emilio Ortega in |
| | | | | the 1900s. Often it is used |
| | | | | for chile relleno. When mature, |
| | | | | it takes on a red color and is |
| | | | | referred to as a Colorado. |
| Banana | Waxy | | 0-500 | Often it is pickled and used as |
| | | | | an ingredient in sandwiches; its |
| | | | | piquancy is not very hot. Its |
| | | | | shape and color resemble a |
| | | | | banana. |
| Bird's Eye | Small hot | Southeast | 50,000-100,000 | A Southeast Asian cultivar |
| | | Asia | | known by many local names, |
| | | | | but generally it is called Thai |
| | | | | chili in the United States. It has |
| | | | | thin fruit with a pointed tip. |
| Black Heart | Ornamental/ | Austria | 5,000-20,000 | Plants can grow up to 1.2 m. |
| | Culinary | | | the flowers are purple. Young |
| | | | | leaves show purple veins, |
| | | | | which may him dark green |
| | | | | later on. The heart-shaped |
| | | | | fruits mature from black to red. |
| Black Hungarian | Ornamental/ | Hungary | 5,000-10,000 | Grows in a conical shape with |
| | Culinary | | | a slight curve near the tip. |
| | | | | Starting from green, they ripen |
| | | | | first to a slightly shiny deep |
| | | | | purple to black which is only |
| | | | | skin deep; when sliced open, |
| | | | | the thick flesh of the interior is |
| | | | | green. As it continues to ripen, |
| | | | | it morphs to red. |
| Cascabel | | Mexico | 3,000 | The small, round fruit are |
| | | | | usually dried, and have a |
| | | | | distinct, nutty flavor. The |
| | | | | name, Spanish for “rattle” or |
| | | | | “jingle bell”, derives from the |
| | | | | rattling noise made by the |
| | | | | seeds inside the dried pod. |
| Cayenne (Red) | Cayenne | French | 30,000-50,000 | This long, thin fruit was |
| | | Guiana | | transported by the Portuguese |
| | | | | to China and India, where it is |
| | | | | used widely. Often it is dried |
| | | | | and ground into powder. |
| Cherry | Pimiento | | 3,500 | Named for the fruit it |
| | | | | resembles, this cultivar's fruit |
| | | | | is small, red, and round. It is |
| | | | | typically used fresh, or pickled |
| | | | | and jarred, and is often used to |
| | | | | stuff green olives. It is also |
| | | | | called pimento. |
| Cheongyang | long, hot | Korea | 10,000 | a medium-sized chili cultivar, |
| | | | | named |
| | | | | after Cheongsong and Yeongy |
| | | | | ang Counties hybrid of |
| | | | | local Jejudo chili with Bird's |
| | | | | eye chili. |
| Chilaca | Pasilla | Mexico | 1,000-2,000 | Popular in Mexican cuisine, it |
| | | | | is almost always encountered |
| | | | | dried; in this state, it is referred |
| | | | | to as a pasilla. The pasilla has a |
| | | | | dark brown color and a smoky |
| | | | | flavor. |
| Chiltepin | Chiltepin | Mexico | 50,000-100,000 | This small, hot fruit is often |
| | | | | eaten by birds. The plant is |
| | | | | thought to be the ancestor of |
| | | | | cultivated C. annuum peppers. |
| | | | | Evidence indicates it has been |
| | | | | consumed by humans as far |
| | | | | back as 7,500 BC. |
| Chimayo | | US | 4,000-6,000 | |
| Cubanelle | | | 1-1,000 | Medium in thickness, the |
| | | | | tapered fruit is green when |
| | | | | unripe, but turns red when |
| | | | | mature. Often it is fried in |
| | | | | Italian cooking. |
| Dangjo | | Korea | | Light green or bright yellow |
| | | | | chili peppers with mild heat. |
| De árbol | | Mexico | 15,000-30,000 | This slender-fruited cultivar is |
| | | | | grown primarily in Mexico. |
| Facing Heaven | Pimiento | China | 30,000-50,000 | |
| Fish | | | 5,000-30,000 | |
| Fresno | Fresno | US | 2,500-10,000 | Similar to the jalapeño, but |
| | | | | with thinner walls, it is |
| | | | | generally used ripe, and has a |
| | | | | higher vitamin content. |
| | | | | Frequently it is used in |
| | | | | ceviche, and is one of the most |
| | | | | frequently used chilis in salsa. |
| Friggitelli | Waxy | Italy | 100-500 | Sweet-tasting and mild, used |
| (Peperoncini) | | | | extensively in Italian and |
| | | | | Greek cuisine, very frequently |
| | | | | pickled. |
| Guntur Sannam | | | 35,000-40,000 | It is well known as a |
| | | | | commercial crop used as a |
| | | | | condiment, culinary |
| | | | | supplement, or vegetable. |
| Hungarian Wax | Waxy | | 2,500-8,000 | This wide, medium-hot variety |
| | | | | is used in Hungarian cuisine, |
| | | | | frequently pickled, dried, |
| | | | | ground, and presented as |
| | | | | “paprika”. |
| Italian Sweet | Long, sweet | Italy | | Used in Spanish cuisine |
| Jalapeño | Jalapeño | Mexico | 2,500-8,000 | Very popular, especially in the |
| | | | | United States, it is often |
| | | | | pickled or canned. A smoke- |
| | | | | dried ripe jalapeño is referred |
| | | | | to as a chipotle. |
| Korean chili | | Korea | 1,500 | Also known as “Korean Dark |
| | | | | Green”, “Korean Long Green”, |
| | | | | “Korean Red” or “Korean Hot” |
| Medusa | Ornamental | | | It is a sweet, ornamental chili |
| | | | | pepper which grows upright |
| | | | | and has brightly colored fruit. |
| Mirasol | | Mexico | 2,000-5.000 | The dried form of the Mirasol |
| | | | | chili is called guajillo, and is |
| | | | | used to make a red sauce used |
| | | | | for tamales. |
| NuMex peppers | Ornamental | US | | Developed by the Chile Pepper |
| | | | | Institute at New Mexico State |
| | | | | University |
| Peter Pepper | Ornamental | US and | 5,000-30,000 | Rare, heirloom-type hot pepper |
| | | Mexico | | cultivated for its unique shape. |
| Peperoncino | Cayenne | Italy | 15,000-30,000 | Generic Italian name for hot |
| | | | | chili peppers, specifically the |
| | | | | cultivars of the species C. |
| | | | | annuum and C. frutescens. |
| Pequin (or piquin) | Small Hot | Mexico | 100,000-140,000 | |
| Piment d'Espelette | Pimiento | Basque | 1,500-2,500 | Fresh fruits, plants and seeds |
| | | Country | | are known as “Gorria”, dried |
| | | (French part) | | fruits are called “Piment |
| | | | | d'Espelette”. “Gorria” is the |
| | | | | Basque word for “red”. Grown |
| | | | | in Espelette since ca. 1650. |
| Padrón | Pimiento | Spain | 500-5,000 | From Galicia. Most are mild |
| (or pimientos de | | | | and very tasty, though about 1 |
| Herbón) | | | | out of 10 may be quite hot. |
| Poblano | Poblano | Mexico | 1,000-2,000 | The large, heart-shaped, dark |
| | | | | green fruit is extremely |
| | | | | popular in Mexico. When |
| | | | | harvested early and dried, it is |
| | | | | referred to as an ancho, When |
| | | | | fully mature and dried, it is |
| | | | | referred to as a mulato. |
| Prik Kee Nu | Small Hot | Thailand | 50,000-100,000 | One of many cultivars called |
| | | | | Thai pepper, it has very short |
| | | | | fruit, and is very hot. |
| Puya (or Pulla) | | Mexico | 5,000 | C. annuum L., hot, medium- |
| | | | | size, green to red, and tapered. |
| Santa Fe Grande | Fresno | | | The Santa Fe Grande is a very |
| | | | | prolific variety used in the |
| | | | | Southwestern United States. |
| | | | | The conical, blunt fruits ripen |
| | | | | from greenish-yellow, to |
| | | | | orange-yellow to red. The |
| | | | | peppers grow upright on 24- |
| | | | | inch plants. Santa Fe Grande |
| | | | | has a slightly sweet taste and is |
| | | | | fairly mild in pungency. |
| Serrano | Serrano | Mexico | 10,000-23,000 | The thin, tapered fruit turns red |
| | | | | when mature. Due to its thin |
| | | | | skin, it does not need to be |
| | | | | peeled before use. |
| Shishito | | Japan | | |
| Siling Mahaba | | Philippines | | A chili pepper grown in the |
| | | | | Philippines, and a popular |
| | | | | ingredient in Filipino Cuisine |

Capsicum baccatum

| | | | |
|---|---|---|---|
| Bishop's Crown | Barbados | 10,000-30,000 | Medium hot pods have a |
| | | | unique shape which resembles |
| | | | the hat of a bishop. Sturdy |
| | | | plants, can be grown as |
| | | | perennials. Also known as |
| | | | bishop's hat, orchid, ají flor, |
| | | | monk's hat. |
| Lemon Drop | | 30,000-50,000 | Very productive variety. Pods |
| | | | are thin walled and have a |
| | | | fruity taste with medium heat. |
| Peppadew | | 1,000-2,000 | Mild, sweet and tangy flavour, |
| | | | usable in many dishes |

Capsicum chinense

| | | | | |
|---|---|---|---|---|
| Adjuma | | | 100,000-500,000 | Very hot, originally cultivated |
| | | | | in Suriname |
| Ají Dulce | | | 0-50 | It is a type of seasoning pepper. |
| | | | | Related to the habanero but |
| | | | | with a much milder flavor. |
| Bhut Jolokia (or | | | Up to 1,040,000 | This cultivar originated |
| Ghost pepper) | | | | in Northeast India, and former |
| | | | | World Records hottest pepper. |
| | | | | It is an interspecific hybrid, |
| | | | | largely C. chinense with |
| | | | | some C. frutescens genes. |
| Carolina Reaper | | US | 1,569,300-2,200,000 | Extremely hot pepper, currently |
| | | | | the Guinness book of world |
| | | | | records holder (Aug. 7, 2013). |
| Datil | | US | 100,000-300,000 | A very hot chili; primarily |
| | | | | grown in Florida |
| Fatalii | | Africa | 125,000-325,000 | Grown in central and southern |
| | | | | Africa, it is very similar in |
| | | | | appearance to and often |
| | | | | confused with the devil's |
| | | | | tongue habanero. |
| Habanero | | | 100,000-350,000 | Subtle, fruity flavor and a floral |
| | | | | aroma. It is closely related to |
| | | | | many other very hot peppers. |
| Hainan Yellow | | Hainan, | 300,000 | Also known as the yellow |
| Lantern | | China | | emperor chili. |
| Infinity chili | | | 1,176,182 | |
| Madame Jeanette | Suriname | Suriname | 100,000-350,000 | |
| Naga Morich | | Bangladesh | 1,000,000 | |
| | | and India | | |
| Naga Viper | | England | 1,382,118 | |
| NuMex Suave | | US | | Developed by the Chile Pepper |
| Orange | | | | Institute at New Mexico State |
| | | | | University. |
| Red Savina | | US | 200,000-580,000 | |
| Scotch Bonnet | | | 150,000-325,000 | Named because of its |
| | | | | resemblance to a Tam o' |
| | | | | Shanter, closely related to the |
| | | | | habanero and is similarly hot. |
| | | | | Often used in Caribbean |
| | | | | cuisine. |
| Trinidad moruga | | | Up to 2,000,000 | Former World-record hottest |
| scorpion | | | | chili. |
| Trinidad Scorpion | | | Up to 1,400,000 | Former world-record hottest |
| ‘Butch T’ | | | | chili. |

Capsicum frutescens

| | | | |
|---|---|---|---|
| African Birdseye | | 50,000-175,000 | Common in Portuguese, |
| (piri piri) | | | Mozambican, and Angolan |
| | | | cuisines. |
| Kambuzi | Malawi | | A small, round cultivar found |
| | | | in central region of Malawi. |
| Siling Labuyo | | 80,000-100,000 | A small, cone-shaped cultivar |
| | | | grown in the Philippines. |
| Tabasco | | 30,000-50,000 | Used in Tabasco sauce. The |
| | | | fruit used only when it ripens to |
| | | | a particular red color. |

Capsicum pubescens

| | | | |
|---|---|---|---|
| Canário | Peru | 30,000-50,000 | A medium hot variety. Thick |
| | | | walled pods are dark yellow |
| | | | when fully ripe and have the |
| | | | size of a small apple. Thrives |
| | | | well under cool growing |
| | | | conditions and can be grown as |
| | | | a perennial. |
| Rocoto (or | Peru, | 30,000-100,000 | Fruit is apple-shaped, with |
| Manzano or | Bolivia | | black seeds. |
| Locoto) | | | |

Fruit of various species of Capsicum contain many classes of phytochemicals with important and/or interesting pharmacological activity. These include but are not limited to polyphenols, flavonoids, carotenoids, ascorbic acid, free radical scavengers, and capsaicinoids. In some embodiments of the present disclosure, the selection of the species of Capsicum to include in the formulation is based on the polyphenol, flavonoid, carotenoid, antioxidant/free radical scavenger, and capsaicinoid content of the tissue.

Compounds known as capsaicinoids cause the spicy flavor (pungency) of chili pepper fruit. There is a linear correlation between concentration of capsaicinoids and Scoville scale (SHU). Capsaicinoid content of a chili pepper may vary during ripening. The primary capsaicinoid in chili pepper is capsaicin, followed by dihydrocapsaicin, nordihydrocapsaicin, homodihydrocapsaicin and homocapsaicin. Capsaicin and dihydrocapsaicin account for approximately 90% of capsaicinoids in chili pepper fruit, are the two most potent capsaicinoids and their molecules differ only in the saturation of the acyl group. Capsaicin (trans-8-methyl-N-vanillyl-6-nonenamide) is a crystalline, lipophilic, colorless and odorless alkaloid with the molecular formula C₁₈H₂₇NO₃. Its molecular weight is 305.40 g/mol, and it is fat-, alcohol- and oil-soluble. Capsaicin displays cis/trans isomerism because the double bond prevents internal rotation. Capsaicin is always found as the trans isomer because in the cis form, the —CH(CH₃)₂ and the longer chain on the other side of the double bond will be close together, causing them to repel each other slightly; this steric hindrance does not exist in the trans isomer.

Capsaicin can bond to transient receptor potential vanilloid 1 (TRPV1), previously known as the vanilloid receptor, which is mainly expressed in the sensory neurons. This receptor is a non-selective, ligand-operated cationic channel located primarily in the small fibers of nociceptive neurons. TRPV1 is also broadly distributed in tissues of the brain, bladder, kidneys, intestines, keratinocytes of epidermis, glial cells, liver, and polymorphonuclear granulocytes, mast cells, and macrophages. It couples with a non-specific cation channel permeable to sodium and calcium ions, and is located in the plasma membrane and the endoplasmic reticulum where regulates intracellular calcium levels. This channel can be regulated and activated by endogenous substance as endovanilloids and diverse exogenous stimuli including chemical agonists as capsaicin, olvanil and resiniferatoxin, ligands highly lipophilic that share structural similarity to several endogenous fatty acids identified as TRPV1 agonists. Some compounds are being used as antagonists for TRPV1, including capsazepine, iodo-resiniferatoxin, ruthenium red, A-425619, SB-366791, AMG9810, and SB-705498. The TRPV1 contains a heat-sensitive subunit responsible for the burning sensation caused by capsaicin. Bonding of capsaicin to TRPV1 increases intracellular calcium, triggering release of neuropeptides such as substance P and the calcium gene-related peptide. Contact between capsaicin and sensory neurons produces pain, inflammation and a localized heat sensation. When applied locally to skin, it promotes an analgesic response due to desensitizing of the sensory neurons caused by substance P depletion. This mechanism has served as a base for studies of the structure-activity relationship which are aimed at development of new synthetic ligands for the TRPV1. Capsaicin's effects in the nervous system are not exclusively analgesic. A number of studies include results showing that it participates in release of somatostatine, CGRP and endotheline.

### D. Other Active Ingredients

In some embodiments, the formulation includes one or more added essential oils selected from Sweet Orange (Citrus sinensis spp), Peppermint (Mentha piperita spp), Lemon (Citrus limon spp), Lavender (Lavendula angustifolia spp) and Vanilla (Vanilla planifolia spp). In some embodiments, the cannabis oil extract includes Vitamin E and one or more essential oils selected from Sweet Orange (Citrus sinensis spp), Peppermint (Mentha piperita spp), Lemon (Citrus limon spp), Lavender (Lavendula angustifolia spp) and Vanilla (Vanilla planifolia spp).

Other essential oils that can be used in the compositions of the invention include, but are not limited to: Agarwood; Agarwood Attar; Ahibero; Allspice; Almond, bitter; Amber Oil; Ambrette Seed; Amyris; Angelica Root; Angelica Seed; Aniseed; Anise; Anise (star); Armoise (Mugwort); Artemisia vestita; Asafoetida; Bakul; Balsam of Peru Oil; Balsam of Peru Resin; Balsamite; Baobab Oil; Basil, Sweet ct Linalool; Basil, Sweet ct Linalool—Organic; Basil, Sweet ct Methyl Chavicol—Organic; Bay; Beeswax; Bergamot; Birch; Boldo; Boronia; Black Cumin; Black Currant Bud; Blue Lotus Attar; Broom; Buchu; Bupleurum (Bupleurum fruticosum); Buddha wood; Butter; Cabreuva; Cade; Cajuput; Calamus; Calendula; Camomile (or Chamomile); Camphor; Cananga; Cangerana; Cape Chamomile (Ericephalus punctulatus) S. Africa, Wild Harvest; Cape May; Caraway; Caraway; Cardamom; Carnation; Carrot Seed; Cascarilla; Cassia; Cassie; Catnip; Cedar (Cedrus) India; Cedarwood; Cedarwood, Atlas-Organic; Cedarwood, Himalayan; Cedarwood, Texas; Cedarwood, Virginia; Celery leaf; Celery Seed; Chamomile, Blue; Chamomile; Chamomile, Roman (Anthemis nobilis); Champa Attar (Michelia champaca) India; Champaca; Chaste tree; Cilantro; Cinnamon; Cinnamon Bark; Cistus; Cistus (Cistus ladaniferus) Corsica; Citronella; Clary Sage Absolute; Clary Sage, Bulgaria; Clary Sage, Russia; Clary Sage, USA; Clementine; Clove; Clove Bud; Cacao; Coconut Pulp; Coffee Bean Oil; Cognac, Green; Coleus; Combava (fruit or leaf); Copaiba; Coriander; Coriander Seed; Cucumber Hydrosol; Cumin; Cumin Seed; Cypress Leaf; Cypress, Blue; Davana; Dill; Elemi; Eucalyptus, Blue Gum; Eucalyptus, Blue Mallee; Eucalyptus, Lemon; Fennel (Foeniculum vulgare) Bulgaria; Fennel, Sweet; Fenugreek; Fern (sweet); Fleabane; Fir Needle; Fir, Balsam; Fir, Douglas; Fir, Silver; Fragonia; Frankincense, India; Frankincense, Somalia; Frankincense Frereana; Frankincense, Oman; Frankincense, Oman; Frankincense, Somalia; Galangal; Galbanum; Geranium; Geranium, Egypt; Geranium, Rose; Geranium, South Africa; Ghandi root; Ginger; Ginger Lily; Ginger, Fresh; Gingergrass (Cymbopogon martinii); Goldenrod; Grapefruit, Pink; Grapefruit, Ruby Red; Grapefruit, White; Hay; Helichrysum, Albania; Helichrysum, Croatia; Hina Attar, India; Hop; Hyssop Decumbens; Hyssop; Immortelle; Jasmine Absolute, Egypt; Jasmine Absolute, India; Jasmine Concrete; Jasmine; Jasmine Sambac; Jatamansi, (Nardostachs jatamansi); Juniper; Juniper Berry (Juniperus communis); Juniper Leaf/Berry; Kaffir Lime; Kava; Labdanum; Larch needle; Laurel (Laurus nobilis) Corsica; Laurel Leaf; Lavandin, Grosso; Lavender—High Elevation; Lavender—Wild; Lavender Absolute; Lavender Hydrosol; Lavender, Bulgaria; Lavender, France; Lavender, Maillette; Leleshwa; Lemon; Lemon Tea Tree; Lemon verbena; Lemongrass; Lentisque (Pistacia lentiscus) Corsica; Lime; Lime Essence Oil; Lime, Distilled; Liquidambar (Styrax); Longoza; Lotus Absolute, Pink; Lotus Absolute, White; Lovage leaf, Lovage root; Magnolia flower; Mandarin; Mandarin, Green; Mandarin, Red; Mandarin, Yellow; Mango ginger; Marjoram; Manila oil; Melissa; Mint; Mint, Himalayan (Mentha arvensis); Mitti Attar; Motia Attar (Jasmine sambac) India; Mugwort; Mustard; Myrrh; Myrtle, Green; Myrtle (Myrtus Communis); Nagarmotha (Cypriol); Neem (Azadirachta indica) India; Neroli; Niaouli; Nutmeg; Nut grass; Oakmoss Absolute; Oakwood; Opopanax, Sweet Myrrh (Commiphora guidotti); Orange, Blood; Orange, Sweet; Orange, Wild; Orange Blossom; Orange Essence Oil; Orange, Bitter Green; Orange, Bitter Red; Oregano; Orris Butter; Osmanthus Absolute; Palmarosa; Palmarosa, Nepal; Palmarosa, Sri Lanka; Palo Santo (Bursera graveolens); Palo Santo; Patchouli; Absolute; Patchouli, Dark; Patchouli, Light; Patchouli, Sri Lanka; Pennyroyal; Pepper, Black; Peppercorn, Pink; Peppermint, Chocolate; Peppermint, France; Peppermint, India; Peppermint, USA; Petitgrain Absolute; Petitgrain Bigarade; Petitgrain sur Fleurs; Petitgrain, Mandarin; Pimento; Pine; Pinion Juniper Co-distillation, Colorado, Wild Harvest; Pinon Pine (Pinus edulis) Colorado, Wild Harvest; Pitta blend (Lavender, Rose Geranium, Ruh Khus); Plai; Pomegranate Seed; Rhododendron (Rhododendron anthopogon); Rhododendron Leaf; Rosalina; Rose; Rose Attar; Rose de Mai Absolute; Rose de Mai Concrete; Rose de Mai Organic Extract; Rose geranium; Rose Hip Seed; Rose Otto, Bulgaria; Rose Otto, Turkey; Rose Otto, White-Organic; Rose vetiver; Rosemary Antioxidant; Rosemary ct Cineole; Rosemary ct Verbenone; Rosewood; Rue; Ruh Khus (Vetiveria zizaniodes); Saffron Attar, India; Sage; Samphire (Cristhmum maritimum) Corsica; Sandalwood; Sandalwood, New Caledonia; Sandalwood, Australian-Premium; Sandalwood (Santalum spicatum), Australia; Sandalwood Oil, Royal Hawaiian (Santalum paniculatum); Sandalwood, Royal Hawaiian; Sassafras; Savitri Rose Perfume; Sea Buckthom; Seaweed; Sierra Juniper (Juniperus occidentalis); Spearmint; Spearmint (Mentha Spicata) Israel; Spikenard; Spikenard, Green; Spruce, Black; Spruce (Picea mariana) Canada; St. John's Wort 2; St. John's Wort (Hypericum perforatum) Bulgaria; Tagetes; Tamanu (Foraha) Oil; Tangelo; Tangerine; Tangerine Murcott; Tansy; Tansy, Blue; Tarragon; Tea Tree; Tea Tree (Leptospermum citratum), Lemon Scented; Tea Tree (Melaleuca alternifolia) South Africa; Thuja; Thyme; Thyme ct Linalool; Tobacco; Tonka Bean; Tuberose; Tulsi, Holy Basic Oil (Ocimum sanctum); Turmeric; Vanilla; Vanilla Bourbon; Verbena; Vetiver-Double Distilled; Vetiver, El Salvador; Vetiver, Haiti; Vetiver, Sri Lanka; Violet Leaf; White Fir (Abies concolor); White Lotus Attar; White Sage (Salvia apiana); Wild Carrot, Corsica; Wintergreen; Wintergreen; Yarrow; Yarrow, Blue; Ylang; Yuzu; and combinations thereof.

The compositions of the invention can also include one or more herbal extracts of Abas, Abele, Abies balsamea, Absinthe, Absinthium, Acacia, Acacia spp., Acai Berries, Acerola, Achillea Millefolium, Achiote, Aconite, Aconitum Napellus, Acorns, Acorus calamus, Acorus gramineus, Adansonia digitata, Adder's Mouth, Adderwort, Adiantum capillus-veneris, Aesculus Hippocastanum, Aframomum melegueta, African Geranium, African Ginger, Agastache foeniculum, Agave, Agnus Castus, Agrimonia Eupatoria, Agrimony, Agropyron Repens, Ague Grass, Ague Root, Ague Tree, Agueweed, Ajamoda, Ajave Seeds, Ajenjo, Ajowan, Ajuga Reptans, Ajvain, Ajwan, Ajwain, Akebia, Akebia quinata, Alaskan Ginseng, Alchemilla Vulgaris, Alchomea Species, Alder, Alder Buckthom, Alder Dogwood, Alecost, Alehoof, Aletris, Aletris Farinosa, Alexandrian Laurel, Alexandrian Senna, Alfalfa, Algarroba, Alkanet, Allheal, Alligator Pepper, Allium cepa, Allium porrum, Allium sativum, Allium schoenoprasum, Allium tuberosum, Allspice, Almond, Alnus glutinosa, Alnus rubra, Aloe ferox, Aloeroot, Aloes, Aloe Vera, Aloysia triphylla, Alpine Strawberry, Alpinia Officinarum, Althaea, Althaea Officinalis, Aluka, Alumroot, Amara Aromatica, Amaracus, Amaranth, Amaranthus Hypocondriacus, Amber Touch-and-heal, Ambroise, Ambrose, Amburana, America-Hodoimo, American Aloe, American Angelica, American Ash, American Aspen, American Basswood, American Bayberry, American Bee Balm, American Beech, American Bugleweed, American Carob, American Cranesbill, American Cress, American Dill, American Dogwood, American Ginseng, American Ground Lily, American Groundnut, American Linden, American Mandrake, American Melissa, American Saffron, American Sanicle, American Sarsaparilla, American Sloe, American Spikenard, American Upland Cotton, American Valerian, American Winter Cress, American Wormroot, American Wormseed, Amla, Ammi Visnaga, Anacardium Occidentale, Ananas Comosus, Anchusa Officinalis, Andiroba, Andrographis, Andrographis paniculata, Anemone, Anemone Pulsatilla, Anemopsis californica, Anethum Graveolens, Angelica, Angelica Archangelica, Angelica Sinensis, Angelica Tree, Angostura, Angostura trifoliata, Anise, Aniseed, Aniseed Stars, Anise Fern, Anise Hyssop, Anise Plant, Annatto, Annona muricata, Annona reticulata, Annual Marjoram, Anthemis Nobilis, Anthoxanthum nitens, Anthriscus cerefolium, Antilles Cherry, Apios americana, Apium Graveolens, Apple, Apple Mint, Apple-of-Peru, Apricot Vine, Apsidium, Aralia Racemosa, Arbe a suif, Arberry, Arboloco, Arbor Vitae, Arbutus, Arbutus menziesii, Arbutus Uva Ursi, Archangel, Archangelica, Archangelica officinalis, Arctium Lappa, Arctostaphylos Uva Ursi, Ardraka, Argan, Argania, Argania spinosa, Argemone Mexicana, Argentine, Aristolochia serpentaria, Aristotelia chilensis, Aritha, Arjaka, Arjuna, Armoracia Rusticana, Armstrong, Amica, Arnica Flowers, Amica Montana, Amica Root, Aromatic Sumac, Aromatic Wintergreen, Arrowroot, Artemisia, Artemisia Abrotanum, Artemisia Absinthium, Artemisia capillaris, Artemisia Dracunculus, Artemisia Tridentata, Artemisia Vulgaris, Artocarpus altilis, Artocarpus heterophyllus, Arugula, Asafoetida, Asclepias Tuberosa, Ascophyllum nodosum, Ash, Ashwaganda, Asian Ginseng, Aspalathus Linearis, Asparagus Cochinchinensis, Asparagus racemosus, Asparagus Root, Asperula Odorata, Aspilia, Aspilia Mossambicensis, Ass-ear, Asthma Plant, Asthma Weed, Astragalus, Astragalus Membranaceus, Atropa belladonna, Auld Wife's Huid, Autumn Crocus, Avena Sativa, Avens, Averrhoa carambola, Avocado, Ayak Chichira, Ayuk Willku, Azadirachta Indica, Azafran, Babchi Seeds, Bacc, Bachelor's-button, Bacopa Monniera, Bahama Berry, Baical Skullcap, Bai Guo, Bai Mu Erh, Ba Ji Tian, Baldina, Balinghoy, Ballota nigra, Balm Mint, Balm of Gilead, Balmony, Balsam Copaiba, Balsam Fir, Balsam of Gilead, Balsam of Peru, Balsam Tree, Bank Cress, Banisteriopsis caapi vine, Baobab, Baptisia, Baptisia Tinctoria, Barbados Aloe, Barbados Cherry, Barbarea verna, Barberry, Barbary Fig, Bardana, Barley, Barosma Betulina, Barren Strawberry, Baran, Basil, Basil Thyme, Basin Sagebrush, Basketbush, Basswood, Bastard Cardamom, Bastard Saffron, Bast Tree, Bauple Nut, Bayawas, Bayberry, Bayberry Bush, Bayberry Wax Tree, Bay Laurel, Beaked Parsley, Bean of India, Bean Trefoil, Bearberry, Bearbind, Beard Lichen, Bear's Foot, Bear's-grape, Bear's-paw, Bear's Weed, Beaumont Root, Beauty Leaf, Bee Balm, Bee Bread, Beech, Beechdrops, Beech Wheat, Bee Plant, Bee Sage, Bee's Nest, Beggar's Buttons, Belladonna, Belle Isle Cress, Bellyache Root, Benjamin Bush, Benzoin Gum, Benzoin Tree, Berberidis, Berberis Aquifolium, Berberis Vulgaris, Berberry, Bergamot Mint, Bergamot Orange, Bertholletia Excelsa, Betel, Bethroot, Betony, Betula alba, Betula pendula, Bhang, Bian Xu, Bible Hyssop, Bible-leaf, Big Sagebrush, Bilberry, Billy-goat Clover, Biltmore Ash, Bindweed, Bird's-foot, Bird's Nest, Birthroot, Birthwort, Biscuits, Bishop's Weed, Bistort, Bitter Aloe, Bitter Ash, Bitter Bark, Bitter Dock, Bitter Leaf, Bitter Melon, Bitter Nightshade, Bitter Orange, Bitter Orange Peel, Bitter Quassia, Bittersweet, Bitter Trefoil, Bitter Wood, Bitterworm, Bixa orellana, Black Alder, Black Alder Tree, Blackberry, Black Cherry, Black Choke, Black Chokeberry, Black Cohosh, Black Cohush, Blackcurrant, Black Dogwood, Black Ginger, Black Haw, Black Henbane, Black Horehound, Black Locust, Black Mustard, Black Pepper, Black Root, Black Sampson, Black Sanicle, Black Snakeroot, Black Stinking Horehound, Black Tany, Blackthorn, Black Thyme, Black Walnut, Black Whortleberry, Blackwort, Bladder Cherry, Bladder Fucus, Bladderpod, Bladderwrack, Blazing Star, Blessed Herb, Blessed Thistle, Blind Nettle, Bloodroot, Blood Vine, Bloodwort, Blooming Sally, Blow Ball, Blue Balm, Blueberry, Bluebottle, Blue Cohosh, Blue Curls, Blue Dandelion, Blue Flag, Blue Giant Hyssop, Blue Ginseng, Blue Gum, Blue Gum Tree, Blue Iris, Blue Mountain Tea, Blue Pimpernel, Blue Rocket, Blue-Sailors, Blue Skullcap, Blue Violet, Blunt-leaved Dock, Bodhi Tree, Bofareira, Bogbean, Bo He, Bola, Boldina, Boldo, Boldoa, Boldu, Boldus, Boneset, Bookoo, Borage, Borago Officinalis, Boswellia carteri, Bo-Tree, Bottlebrush, Bouncing Bet, Bourtree, Bowman's Root, Boxberry, Boxwood, Brahmi, Bramble, Brandy Mint, Brassica alba, Brassica juncea, Brassica nigra, Brassica oleracea, Brassica rapa Pekinensis, Brazilian Ginseng, Brazil Nut, Breadfruit, Bread Wheat, Bride's Button, Bridewort, Brigham Tea, Brindall Berry, Brindle Berry, Brinton Root, British Myrrh, Broad-leaved Dock, Bromelain, Brook Bean, Brooklime, Broom, Broom Flowers, Broom Tops, Broom Tea-Tree, Brown Mustard, Brownwort, Bruisewort, Bryonia Alba, Bryony, Buchu, Buckbean, Buckeye, Buckler-leaved Sorrel, Buckthorn, Buckwheat, Bucku, Buddha Fruit, Buffalo Herb, Bugbane, Bugbane Squawroot, Bugle, Bugleweed, Bugloss, Bu Gu Zhi, Bugwort, Bull Flower, Bullock's Heart, Bull's Heart, Bunny's Ears, Bupleurum, Bupleurum Chinense, Bur, Burage, Burdock, Burdock Burrs, Burren Myrtle, Burr Marigoldt, Burrs, Burr Seed, Bush Nut, Butcher's Broom, Butterbur, Butterfly Weed, Butternut, Butter Winter, Butterwort, Butterweed, Buttons, Caban Cherry, Cabbage, Cabbage Palm, Cabbage Rose, Cacao, Cacari, Cajeput Tree, Cajueiro, Calabar Bean, Calamint, Calamintha Nepeta, Calamus, Calendula, Calendula Officinalis, California Poppy, Calluna Vulgaris, Calophyllum inophyllum, Caltrop, Calumba, Cambodian Mint, Camel Grass, Cammellia Sinensis, Camocamo, Camphor, Camphor Tree, Camptotheca Acuminata, Camu, Canabis, Canada Balsam, Canada Root, Canada Tea, Canadian Fleabane, Canaigre, Cananga odorata, Cancerosa, Caner Root, Cancer Tree, Candle Berry, Cane Ash, Canistel Fruit, Cankerwort, Cannabis Sativa, Cape Aloe, Cape Gooseberry, Caperberry, Caperbush, Capers, Capon's Tail, Capparis spinosa, Capsaicin, Capsella Bursa-Pastoris, Capsicum, Capsicum Annuum, Capsicum chinense, Carambola, Carapa guianensis, Caraway, Caraway Seed, Cardamom, Cardamom Seeds, Cardamon, Carduus Marianus, Carica Papaya, Carob, Carolina Jasmine, Carom, Carony Bark, Carpenter's-herb, Carpenter's-square, Carpenter's Weed, Carrageen, Carrot, Carthamus Tinctorius, Carum Carvi, Cascara, Cascara Buckthom, Cascara Sagrada, Caseweed, Cashew Nut Shells, Cassava, Cassia Senna, Castanea Sativa, Castor Bean Plant, Castor Oil Plant, Catalonian Jasmine, Catchweed, Catha, Catha Edulis, Catmint, Catnep, Catnip, Catrup, Cat's Claw, Cat's-foot, Cat's-play, Catswort, Cat Thyme, Catuaba, Caulophyllum Thalictroides, Cayenne, Ceanothus Americanus, Cedar Nut, Celandine, Celery, Centaurea Cyanus, Centaurium Erythraea, Centaury, Centella Asiatica, Century Plant, Cephaelis Ipecacuanha, Cerasee, Ceratonia Siliqua, Cereso, Cetraria Islandica, Chaga Mushroom, Chai Hu, Chamaelirium Luteum, Chamomile, Chanca Piedra, Chandan, Chang Pu, Chaparral, Charapilla, Chaste Berry, Chaste Tree, Chat, Chaulmoogra, Checkerberry, Cheeseflower, Cheese Rennet, Cheiranthus Cheiri, Chelidonium Majus, Chelone Glabra, Chenopodium Ambrosioides, Chen-Pi, Cherry Birch, Chervil, Chia, Chian, Chien, Chiang, Chickweed, Chicory, Chih-ma, Chi-hsueh-ts'ao, Chilean Wineberry, Chilgoza, Chili Pepper, Chimaphila Umbellata, China Root, Chin-ch'iao-mai, Chinese Angelica, Chinese Cabbage, Chinese Chives, Chinese Foxglove, Chinese Ginseng, Chinese Gold Thread, Chinese Lantern, Chinese Licorice, Chinese Mustard, Chinese Nettle, Chinese Star Anise, Chinese Wolfberry, Chink, Chionanthus Virginicus, Chirayata, Chiretta, Chittembark, Chives, Chocolate, Chocolate Root, Chocolate Vine, Choke Cherry, Chondrus Crispus, Christmas Tree, Chrysanthemum, Chrysanthemum Balsamita, Chrysanthemum Cinerariifolium, Chrysanthemum Morifolium, Chuan Xin Liang, Chuchupate, Church Steeples, Cicely, Cichorium Intybus, Cilantro, Cimicifuga, Cimicifuga Racemosa, Cinchona, Cinchona Bark, Cinchona spp, Cingulum Sancti Johannis, Cinnabar Root, Cinnamomum Camphora, Cinnamomum Zeylanicum, Cinnamon, Cinnamonwood, Cinquefoil, Cirsium Vulgare, Citroengrass, Citrus aurantium, Citrus bergamia, Citrus ichangensis×Citrus reticulata var. austera, Citrus limon, Citrus reticulata, Citrus thyme, City Avens, Clary, Clary Sage, Clear Eye, Cleavers, Clematis Stem, Clove, Clove Garlic, Clover, Clover Broom, Clove Root, Clown's Woundwort, Clubfoot Moss, Club Moss, Cnicus Benedictus, Coca, Coca Shrub, Cocashweed, Cochlearia Officinalis, Cocklebur, Cockle Buttons, Cocoa, Codonopsis, Codonopsis pilosula, Coffea Arabica, Coffee, Coffeeweed, Coicis, Coix, Coix Lachryma-jobi, Cola nitida, Colchicum, Colchicum Autumnale, Coleus, Coleus Forkolil, Coleus Forskohlii, Colewort, Colicroot, Colla, Collinsonia Canadensis, Colorado Cough Root, Coltsfoot, Colt's-tail, Comfrey, Commiphora Molmol, Commiphora Mukul, Commiphora Opobalsamum, Common Alder, Common Alkanet, Common Anise, Common Arnica, Common Ash, Common Balm, Common Barberry, Common Basil, Common Blue Violet, Common Broom, Common Buckthorn, Common Buckwheat, Common Bugle, Common Burnet, Common Caraway, Common Centaury, Common Chamomile, Common Club Moss, Common Cotton, Common Dock, Common Dill, Common Fennel, Common Fenugrec, Common Flax, Common Foxglove, Common Hazel, Common Holly, Common Hop, Common Horehound, Common Hyssop, Common Jasmine, Common Juniper, Common Lavender, Common Lime, Common Madder, Common Mallow, Common Marjoram, Common Nettle, Common Oats, Common Onion, Common Parsley, Common Periwinkle, Common Privet, Common Rue, Common Sage, Common Sagebrush, Common Sea-Buckthorn, Common Stinging Nettle, Common Strawberry, Common Sundew, Common Thistle, Common Thyme, Common Wheat, Common White Jasmine, Common Willow, Common Wormwood, Compass Plant, Compass Weed, Compositae, Conium Maculatum, Consormol, Consumptive's Weed, Convallaria Majalis, Convolvulus Sepium, Cool Tankard, Copaiba, Copal, Copaifera Species, Coptidis, Coptis, Coptis chinensis, Coptis Rhizome, Cordyceps, Cordyceps sinensis, Coriander, Coriandrum sativum, Corn, Comelian Tree, Cornflower, Cornish Lovage, Corn Mint, Corn Poppy, Corn Rose, Comsilk, Cornus Florida, Corsican Mint, Corsican Pepper, Corydalis, Corydalis Rhizome, Corydalis Yanhusuo, Corylus avellana, Costmary, Cotton, Cotton Thistle, Couch Grass, Coughroot, Coughweed, Coughwort, Countryman's-treacle, Cowbloom, Cow Chervil, Cow Clover, Cow Cress, Cowgrass, Cowplant, Cowslip, Crampbark, Crampweed, Cranberry, Cranberry Bush, Cranberry Tree, Cranesbill, Crataegus Monogyna, Crataegus Oxyacantha, Crataeva, Crataeva nurvula, Cream Of Tartar Tree, Creathnach, Creeping Charlie, Creeping Thyme, Creosote Bush, Crocus sativus, Crosswort, Croton Lechleri, Crowberry, Crow Corn, Cuban Oregano, Cubeb Pepper, Cuckoo's Cap, Cucurbita Pepo, Culantrillo, Culver's Physic, Culver's Root, Cumaru, Cumaruzeiro, Cumin, Cuminum cyminum, Curacao Aloe, Curare, Curcuma longa, Curcuma zedoaria, Cure-all, Curled Dock, Curled Mint, Curly Parsley, Curry-leaf tree, Curry Tree, Cuscus, Cuscuta Epithymum, Cusparia Bark, Custard Apple, Cutleaf Bugleweed, Cutweed, Cydonia Oblonga, Cymbopogon Citratus, Cypripedium Pubescens, Da Huang, Dalcini, Dalmatian Iris, Dalmation Insect Flower, Dalmation Pellitory, Dalmatian Sage, Damiana, Dandelion, Dang Gui, Danish Dill, Dan Shen, Daruharidra, Da suan, Datura Stramonium, Daucus carota, Deadly Nightshade, Deadmen's Bells, Dead Nettle, Dead-Rat Tree, Death-flower, Deerberry, Desert Cactus, Desert Oregano, Desert Tea, Devil's-apple, Devil's Bit, Devil's-bones, Devil's Cherries, Devil's Claw, Devil's Club, Devil's Dung, Devil's-eye, Devil's Guts, Devil's Herb, Devil's Plague, Dew of the Sea, Dhup, Digitalis Purpurea, Di Huang, Dill, Dillisk, Dillseed, Dillweed, Dilly, Dilsk, Dilo Oil Tree, Dioscorea Villosa, Diosma Betulina, Dipsacus Sylvestris, Dipteryx Odorata, Divale, Djamboe, Doda, Dodan, Doadni, Dodder, Dog Brier, Dog Grass, Dog Rose, Dog's Mercury, Dog Tree, Dogwood, Dong Chong Xia Cao, Dong Quai, Dovefoot, Drago, Dragon's Blood, Dragon's mugwort, Dragonwort, Dropsy Plant, Drosera Rotundifolia, Dryopteris filix-mas, Ducks Foot, Dulse, Dutch Clover, Dwale, Dwarf Juniper, Dwarf Nasturtium, Dwayberry, Dyeberry, Dyer's Broom, Dyer's Greenweed, Dyer's Madder, Dye's-saffron, Dysphania ambrosioides, Early Winter Cress, Earth-smoke, Easter Flower, Easter Giant, Eau-de-cologne Mint, Echinacea, Echinacea Angustifolia, Egg Fruit, Egg Wrack, Egyptian Privet, Eight-horned Anise, Eight-horns, Ela, Elaci, Elder, Elder-berry, Elder-flower, Elecampane, Eletteria Cardamomum, Eleuthero, Eleutherococcus Senticosus, Elk Mint, Emetic Herb, Enandi, Endive, English Alder, English Balm, English Catnip, English Chamomile, English Hawthorn, English Holly, English Hop, English Mandrake, English Serpentary, English Thyme, English Valerian, English Violet, English Wallflower, Epazote, Ephedra, Ephedra Nevadensis, Ephedra Sinica, Epifagus virginiana, Epilobium Angustifolium, Epimedium, Epimedium grandiflorum, Equisetum Arvense, Erigeron canadensis, Eriodictyon Califomicum, Eruca vesicaria sativa, Erythraea Centaurium, Erythroxylum Catuaba, Erythroxylum Coca, Eschscholzia Californica, Espinheira Santa, Estragon, Ethiopian Cumin, Eucalyptus, Eucalyptus Globulus, Eugenia Carophyllata, Eupatorium, Eupatorium Perfoliatum, Eupatorium Purpureum, Euphorbia Hirta, Euphrasia Officinalis, European Alder, European Angelica, European Ash, European Barberry, European Black Alder, European Buckthom, European Centaury, European Chestnut, European Cowslip, European Dill, European Elder, European Holly, European Hop, European White Water lily, European Willow, Euterpe Oleracea, Evening Primrose, Evening Star, Evergreen, Eye Balm, Eyebright, Eyeroot, Fagopyrum Esculentum, Fagus Grandifalia, Fah Tolai, Fairy Cup, Fairy's Glove, False Acacia, False Box, False Chamomile, False Jasmine, False Saffron, False Unicorn, False Valerian, False White Cedar, Featherfew, Featherfoil, Feather Geranium, Febrifuge Plant, Felon Herb, Felonwort, Female Fern, Fennel, Fenugreek, Ferula Asafoetida, Fetid Horehound, Fever Bush, Feverfew, Fever Grass, Fever Tree, Feverwort, Fiber, Ficus religiosa, Field Balm, Field Pansy, Field Poppy, Field Pumpkin, Field Sorrel, Figwort, Filipendula Ulmaria, Fir, Fir Balsam, Fireweed, Fir Pine, Fishfuddle, Five-fingers, Five-leaf, Flag Lily, Flagroot, Flanders Poppy, Flannelleaf, Flat-leaved Parsley, Flax, Flax Seed, Flax Weed, Fleabane, Flea Seed, Flesh and Blood, Fleur-de-lis, Flinders Rose, Florentine Iris, Florida Dogwood, Florida Fishpoison Tree, Flower de-luce, Flowering Dogwood, Flowering Willow, Flowery Knotweed, Foeniculum Vulgare, Folk's Glove, Food Of The Gods, Forsythia, Forsythia suspensa, Fo Ti, Foxberry, Fox Geranium, Foxglove, Fox Grape, Foxtail, Fragaria ananassa, Fragaria Vesca, Fragrant Balm, Fragrant Giant Hyssop, Fragrant Sumac, Frankincense, Fraxinus Americana, Fraxinus Excelsior, French Basil, French Lilac, French Parsley, French Rose, French Sorrel, French Tarragon, French Thyme, Friar's Cap, Fringe Tree, Fritillaria, Fritillaria Thunbergii, Frog Plant, Fucus Vesiculosus, Fu-ling, Fuller's-herb, Fumaria officinalis, Fumitory, Gagroot, Galangal, Galega Officinalis, Galipea officinalis, Galium Aparine, Galium Odoratum, Galium Verum, Gambooge, Gan cao, Gandana, Ganja, Gan-jiang, Ganoderma Lucidum, Gao Liang, Garabato, Garcinia, Garcinia cambogia, Garcinia gummi-gutta, Garcinia Kola, Garden Angelica, Garden Balm, Garden Basil, Garden Bumet, Garden Chamomile, Garden Chervil, Garden Chicory, Garden Dill, Garden Heliotrope, Garden Hyssop, Garden Lavender, Garden Loosestrife, Garden Marigold, Garden Mint, Garden Myrrh, Garden or Green Purslane, Garden Patience, Garden Rosemary, Garden Rue, Garden Sage, Garden Thyme, Garden Violet, Garlic, Garlic Chives, Garlic Sage, Gaultheria Procumbens, Ge-gen, Gelsemium Sempervirens, Genista, Genista Tinctoria, Gentian, Gentiana Lutea, Geranium Maculatum, Geranium Robertianum, Geraniums, German Chamomile, Germander, German Mustard, German Rue, German Tarragon, German Thyme, German Valerian, Geum urbanum, Ghaap, Gill Run Over, Ginger, Ginkgo, Ginkgo Biloba, Ginkgo Nut, Ginny Grains, Ginny Papper, Ginseng, Glechoma Hederacea, Glossy Buckthom, Glycine max, Glycyrrhiza Glabra, Goathead, Goat's Rue, Goatweed, Goat Wort, Gold Coin Grass, Golden Aspen, Goldenberry, Golden Flower of Mary, Golden Loosestrife, Golden Ragwort, Golden Root, Goldenrod, Goldenseal, Golden Senecio, Gold Melissa, Goldy Star, Goosefoot, Goose Grass, Goosewort, Gorikapuli, Gospel Tree, Gossypium Hirsutum, Gotu Kola, Gourmet Parsley, Goutweed, Gow Choy, Graines, Grains Of Paradise, Gramineus, Grape, Grape Vine, Grass, Grass Burdock, Gravelroot, Graviola, Graybeard, Greasewood, Great Burdock, Greater Burnet, Greater Cardamom, Great Morel, Great Nettle, Great Stinging Nettle, Great Wild Valerian, Greek Hayseed, Green Ginger, Green Ozier, Green Tea, Grifola Frondosa, Grindelia, Grindelia Camporum, Groats, Ground Berry, Ground Cherry, Ground Holly, Ground Ivy, Ground Juniper, Ground Lemon, Ground Lily, Groundnut, Ground Pine, Ground Raspberry, Grouse Berry, Guaiac, Guaiacum, Guajacum, Guaiacum Officinale, Gualtheria Procumbens, Guarana, Guasai, Guava Tree, Guelder Rose, Guggul, Gui, Guinea Grains, Guinea Pepper, Guinea Seeds, Gum Bush, Gum Guggulu, Gum Myrrh Tree, Gumplant, Gurmabooti, Gurmar, Gymnema, Gymnema sylvestre, Gynostemma, Gynostemma pentaphyllum, Gypsyweed, Gypsywort, Habanero Pepper, Hackmatack, Hai-ts'ao, Hamamelis Virginiana, Handflower, Happy Tree, Hapusha, Hardock, Hareburr, Hare's Ear Root, Harpagophytum Procumbens, Hartshorn Plant, Harvest Lice, Hasabis, Hashish, Hatomugi, Haw, Hawaii Nut, Hawkweed, Hawthorn, Haymaids, Hazelnut Tree, Heal-All, Heart Of The Earth, Heartsease, Heather, Hebanthe Paniculata, Hedge Bind Weed, Hedge Fumitory, Hedge Maid, Hediondilla, Helianthus Annuus, Heliotrope, Hellweed, Helmet Flower, Helonias Root, Hemlock, Hemp, Henbane, Henna, Herb Bennet, Herb-of-Grace, Herb of St. Barbara, Herb of The Angels, Herb-of-the-cross, Herb Robert, Hercules Woundwort, He Shou Wu, Hibiscus, Hibiscus Sabdariffa, Hieracium Pilosella, Hierochloe odorata, High Angelica, High Mallow, Hill Berry, Hina, Hind Heal, Hineheel, Hing, Hini, Hippophae rhamnoides, Hip Rose, Hoarhound, Hock-heal, Hodoimo, Hoelen, Hog Apple, Hog Bean, Hog Cranberry, Hogweed, Holigold, Holly, Holy Basil, Holy Ghost Plant, Holy Herb, Holy Grass, Holy Thistle, Honey Plant, Honeysuckle, Hoodia, Hoodia pilifera, Hood Weed, Hoodwort, Hook-heal, Hopniss, Hops, Hops Vine, Horehound, Horny Goatweed, Horse Balm, Horse Chestnut, Horsefly Weed, Horseheal, Horse Mint, Horseradish, Horse Savin, Horsetail, Horse Thistle, Horseweed, Ho She Wu, Ho Shou Wu, Hot Mint, Hsia-ku-ts'ao, Hsiao-hui-hsiang, Hsieh-tzu-ts'ao, Hua-Hsian, Huang Qi, Huang Quin, Huarango, Hu-chin-ts'ao, Huckleberry, Hu-lu-ba, Hu-lu-pa, Humulus Lupulus, Hungarian Chamomile, Huo Ma Ren, Hurrburr, Hurtleberry, Hurtsickle, Husk Cherry, Hydnocarpus, Hydnocarpus kurzii, Hydrangea, Hydrangea Arborescens, Hydrastis Canadensis, Hyoscyamus Niger, Hypericum, Hypericum Perforatum, Hyssop, Hyssopus Officinalis, Iceland Lichen, Iceland Moss, I-chi-kao, Ignatia Amara, Ignatius Bean, Ilang-Ilang, Ilex Aquifolium, Ilex Paraguariensis, Illicium verum, Ill-scented Sumac, Imburana De Cheiro, Incensier, Indian Apple, Indian Arrowroot, Indian Balm, Indian Balmony, Indian Bedellium, Indian Borage, Indian Bread, Indian Chickweed, Indian Corn, Indian Dye, Indian Elm, Indian Gentian, Indian Ginseng, Indian Gooseberry, Indian Lotus, Indian Mustard, Indian Nettle, Indian Nut, Indian Olibanum, Indian Paint, Indian Pennywort, Indian Pink, Indian Plant, Indian Plume, Indian Potato, Indian Red Paint, Indian Root, Indian Sage, Indian Shamrock, Indian Snakeroot, Indian Tobacco, Indian Tree, Indigo Broom, Inonotus obliquus, Inula Helenium, Ipecac, Ipecac Shrub, Ipio, Iporoni, Iporuru, Iris, Iris Florentina, Iris Germanica, Irish Broom, Iris pallida, Irish Moss, Iris Versicolor, Italian Bumet, Italian Cress, Italian Jasmine, Italian Lovage, Italian Pimpernel, Ivory Plum, Jaborandi, Jackfruit, Jack Tree, Jak, Jacob's Chariot, Jacob's-staff, Jacon, Jamaican Dogwood, Jamaica Pepper, Jamaica Sorrel, Jambu, Jambul, Jamestown Weed, Japanese Catnip, Japanese Grapefruit, Japanese Horseradish, Japanese Mint, Japanese Mushroom, Japanese Seaweed, Jasmin, Jasmine, Jasmini Flos, Jasminum spp., Jateorhiza Palmata, Jaundice Berry, Jaundice Root, Java Pepper, Java Plum, Jersey Tea, Jerusalem Oak, Jessamine, Jesuit's Balsam, Jesuit Tea, Jew's-harp Plant, Jiaogulan, Jicara, Jimsonweed, Jing Jie, Jin Qian Cao, Jin Yin Hua, Job's Tears, Joe Pye Weed, Juglans cinerea, Juglans Nigra, Juglans Regia, Johnny-jump-up, Johnswort, Joint Fir, Ju Hua, Juniper, Juniper Bark, Juniper Berry, Juniper Bush, Jupiter's Bean, Juniperus Communis, Kachur, Kalmegh, Kamoteng Kahoy, Kanma, Kan-ts'ao, Kappa, Katphala, Kava, Kelp, Kelpware, Kemangen, Keyflower, Key of Heaven, Khas, Khat, Khella, Kiawe, Kidney Stone Tree, King Of Bitters, King's-clover, King's Crown, King's Cure, King's-cure-all, Kinnikinnick, Kiryat, Klamath Weed, Knitback, Knitbone, Knotgrass, Knotted Kelp, Knotted Marjoram, Knotted Wrack, Knotty Brake, Knotweed, Kola Nut, Korean Ginseng, Kua-lou, Kuawa, Kudzu, Kuei, Kumari, Kumaru, Kuo-lao, K'u-tou, Ku Ts'ai, Lactucarium, Lactuca Virosa, Ladder-to-heaven, Ladies'-delight, Ladies' Seal, Lady Bleeding, Lady's Bedstraw, Lady's Slipper, Lady's Mantle, Lady's-washbowl, Lai-ei-ts'ao, Lamb Mint, Lamium Album, Lammint, Lang-tu, Langue de Boeuf, Lapacho, Lappa, Lappa Minor, Large Fennel, Large-leaved Germander, Larrea Tridentata, Latherwort, Laurus Nobilis, Lavender, Lavender Giant Hyssop, Lavandula officinalis, Lawn Chamomile, Lawsonia inermis, Leafcup, Lebanese Oregano, Leeks, Lemon, Lemon Balm, Lemon Thyme, Lemon Verbena, Lemongrass, Lentinus Edodes, Leonurus Cardiaca, Leopard's Bane, Lepidium meyenii, Leptandra, Leptandra Virginica, Leptospermum scoparium, Lesser Indian Cress, Lesser Periwinkle, Le-ts'ao, Lettuce Opium, Levisticum Officinale, Lian Qiao, Licorice, Licorice Mint, Licorice Root, Life Root, Lignum Vitae, Ligusticum Porteri, Ligustrum Vulgare, Lily Convalle, Lily of the Valley, Limaosinho, Limeblossom, Lime Flowers, Lime Mint, Lime Tree, Linden, Linden Flower, Lindera benzoin, Ling Chi, Ling-t'ung, Ling Zhi, Link, Linseed, Lint Bells, Linum Usitatissimum, Lion's Ear, Lion's Foot, Lion's Tail, Lion's Tart, Lion's Tooth, Lippia graveolens, Lipstick Tree, Liquorice, Live-Forever, Live-Long, Liver Lily, Liverwort, Lizard's Tail, Lobelia, Lobelia Inflata, Longevity Herb, Lonicera Caprifolium, Lonicera Japonica, Lonicera Spp, Loodroot, Loosestrife, Lophophora williamsii, Lotus, Lotus Comiculatus, Lousewort, Lovage, Love Apples, Love in Winter, Love-lies-bleeding, Love Persley, Love Vine, Lucerne, Lu Hui, Lungwort, Luole, Lychee, Litchi chinensis, Lycium, Lycium Chinense, Lycium Fruit, Lycopodium, Lycopodium clavatum, Lycopus americanus, Lysimachia christinae, Lysimachia vulgaris, Lythrum salicaria, Ma Bian Cao, Macadamia Nut, Macadamia spp., Maca, Maca-Maca, Mace, Macochihua, Madagascar Periwinkle, Mad Apple, Madder, Madder Root, Madderwort, Mad Dog, Madweed, Madrone Tree, Ma huang, Maidenhair Fern, Maidenhair Tree, Maid's-hair, Maino, Maitake, Maize, Maka, Malabar Cardamom, Malabar Plum, Malabar Tamarind, Mal-dos-sete-dias, Male Fern, Mallow, Malpighia species, Malus Communis, Malva Sylvestris, Mamey Sapote, Manac, Mandioc, Mandragora, Mandragora Officinarum, Mandrake, Mangosteen Oil Tree, Manihot esculenta, Manioc, Manioc Root, Manna Grass, Man-t'ien-hsing, Manuka, Manuka Myrtle, Manuka Tree, Manzanilla, Maqui, Maramar, Maranta Arundinaceae, Maranta Starch, Marapuama, Mare's Tail, Marigold, Maroochi Nut, Marrubium, Marrubium Vulgare, Marshmallow, Marsh Marigold, Marsh Parsley, Marsh Trefoil, Marsh Woundwort, Marum, Marvel, Mary Bud, Mary Golde, Mary Gowles, Mary Jane, Maryland Pink, Mary's Grass, Mary's Mantle, Mary's Thistle, Master of the Woods, Masterwort, Mastic, Mate, Matricaria Chamomilla, May, Mayapple, May Blossom, Maybush, May Lily, Maypop, Maytenus, Maytenus Species, May Tree, Meadow Clover, Meadow Eyebright, Meadow Saffron, Meadow Sage, Meadowsweet, Mealberry, Medicago Sativa, Mei-ts'ao, Melaleuca, Melaleuca Altemifolia, Melegueta Pepper, Melilot, Melilotus Officinalis, Melissa, Melissa Officinalis, Melmot Berry, Mentha haplocalyx, Mentha piperita, Mentha pulegium, Mentha requienii, Mentha suaveolens, Mentha spicata, Menthol Mint, Mentha×piperita citrata, Menyanthes Trifoliata, Mercurialis Perennis, Mescal, Meshasringi, Mesquite, Methi, Mexican Mint, Mexican Oregano, Mexican Poppy, Mexican Potato, Mexican Tea, Mexican Thyme, Mexican Wild Yam, Mexico Seed, Middle Comfrey, Mignonette Tree, Mi-kan, Milfoil, Milk Ipecac, Milk Thistle, Milkwort, Millefoil, Mint, Mints, Miracle Herb, Miracle Tea, Mistletoe, Mitchella Repens, Mi-tieh-hsiang, Mi-ts'ao, Moccasin Flower, Mogo, Molina, Mo Li Hua, Momordica Charantia, Monarda Didyma, Monkey-Bread Tree, Monkshood, Monk's Pepper, Moonflower, Moon Grass, Moose Elm, Morinda, Morinda citrifolia, Morinda officinalis, Mormon Tea, Moroccan Ironwood, Mortification Root, Morus Nigra, Mother-of-thyme, Mother's-heart, Motherwort, Moujean Tea, Mountain Arnica, Mountain Ash, Mountain Aspen, Mountain Balm, Mountain Berry, Mountain Box, Mountain Cranberry, Mountain Daisy, Mountain Grape, Mountain Holly, Mountain Mint, Mountain Savory, Mountain Strawberry, Mountain Tobacco, Mountain Tea, Mouse-ear, Mugwort, Muira Puama, Mujonso, Mulberry, Mullein, Murraya koenigii, Muscatel Sage, Mu-Su, Mu Tong, Mu-yao, Myrciaria dubia, Myrica, Myrica Cerifera, Myricae Cortex, Myristica Fragrans, Myroxylon Balsamum, Myroxylon Pereirae, Myrrh, Myrrhis odorata, Myrtle, Myrtle Pepper, Myrtus communis, Nagara, Naidi, Naked Ladies, Napa Cabbage, Nappa, Narrow Dock, Narthex, Nashia inaguensis, Nasturtium Officinale, Naughty Man's Cherries, Neem, Nelumbo nucifera, Nenuphar, Nep, Nepeta Cataria, Nerveroot, Nettle, Nettle Flowers, New England Pine, New Jersey Tea, New Zealand Tea Tree, Niando, Nicotiana Rustica, Nightshade, Night Willow Herb, Nine Hooks, Nine Joints, Nip, Nira, Niu Bang, Noble Chamomile, Noble Yarrow, Nodding Wakerobin, Noni, Normandy Cress, Northern Pine, Northern Spicebush, Northern White Cedar, Norwegian Kelp, Nosebleed, Nutmeg, Nux Vomica, Nymphaea Alba, Oak, Oats, Ocimum basilicum, Ocimum tenuiflorum, Oenothera Biennis, Ohio Curcuma, Oil plant, Oilnut, Oil Nut Tree, Old English Lovage, Old-maid's-nightcap, Old-maid's-pink, Old Man, Old-man's-beard, Old Man's Nightcap, Old Man's Pepper, Old woman, Olea Europaea, Olibanum, Olive, Omam, Omum, One-berry, Onion, Opium Poppy, Oplopanax horridus, Orange, Orange Mint, Orange Root, Ordeal Bean, Oregano, Oregano Brujo, Oregon Grape, Oriental Garlic, Oriental Mustard, Oriental Poppy, Origanum majorana, Origanum syriacum, Origanum vulgare, Orpine, Orris Root, Osha, Osterick, Oswego Tea, Our-Lady's-bedstraw, Our-Lady's-tears, Oval Buchu, Owler, Oxadoddy, Ox Balm, Ox Heart, Ox-tongue, Pacific Madrone, Pacific Yew, Padmaka, Paeonia Officinalis, Paico, Paigle, Palisade Pine, Palma Christi, Palmaria Palmata, Palmetto, Panax Ginseng, Panax Quinquefolium, Panay, Pansy, Papaver orientale, Papaver Rhoeas, Papaver Somniferum, Papaya, Paper Birch, Papoose Root, Paracress, Paraguay Tea, Pareira, Parell, Parietaria Officinalis, Pariswort, Parsley, Parsnip, Partridgeberry, Pasque Flower, Passiflora, Passiflora Incamata, Passion Flower, Passions, Passion Vine, Pastinaca sativa, Patchouli, Patience Dock, Patience Herb, Pau d'Arco, Paullinia Cupana, Pausinystalia Yohimbe, Pauson, Peepal Tree, Pei-ma, Peking Cabbage, Pelargonium, Pelargonium sidoides, Pelargonium species, Pellitory, Pellitory Of The Wall, Pembina, Pennyroyal, Peony, Pepperidge Bush, Peppermint, Pepperweed, Perfume Tree, Periploca Of The Woods, Persea Americana, Persian Berries, Persicaria odorata, Personata, Peruvian Balsam, Peruvian Bark, Peruvian Ginseng, Peruvian Ground Cherry, Petasites Hybridus, Petokal, Petroselinum Crispum, Peumus Boldus, Peyote, Pfaffia paniculata, Philanthropos, Phudina, Phyllanthus, Phyllanthus emblica, Phyllanthus niruri, Physalis Alkekengi, Physalis Peruviana L., Physic Root, Physostigma venenosum, Phytolacca Americana, Picrasma Excelsa, Pigeon's Grass, Pignoli, Pigweed, Pikake, Pill-Bearing Spurge, Pilocarpus Microphyllus, Pilosella, Pimbina, Pimenta Dioica, Pimenta Officinalis, Pimpinella Anisum, Pine, Pine Nut, Pineapple, Pineapple Strawberry, Pineapple Verbena, Pinkroot, Pink Rose, Pinon Nut, Pinus spp., Pinus Strobus, Pinus Sylvestris, Pinyon Pinenut, Piper betle, Piper cubeba, Piper Methysticum, Piper Nigrum, Pipe Tree, Pipsissewa, Pistachio, Pistacia vera, Piscidia piscipula, Pissabed, Pistachio, Plantago Major, Plantago Psyllium, Plantago Seed, Plantain, Plectranthus amboinicus, Pleurisy Root, Plum Rose, Podophyllum Peltatum, Poet's Jasmine, Pogostemon cablin, Poha Berry, Poison Ash, Poison Flag, Poison Parsley, Poison Tobacco, Pokeroot, Pokeweed, Polar Plant, Polygala Senega, Polygonatum Multiflorum, Polygonum Aviculare, Polygonum Bistorta, Polygonum Multiflorum, Polygonum odoratum, Polypodium Vulgare, Polypody, Poor Man's Ginseng, Poor-man's-treacle, Poplar, Popotillo, Populus alba, Populus Tremuloides, Poria, Poria cocos, Portulaca Oleracea, Pot, Potato Bean, Potency Wood, Potentilla Anserina, Potentilla Erecta, Potentilla Reptans, Potenzholz, Pot Marigold, Pouteria sapota, Pouteria campechiana, Prairie Smoke, Prickly Ash, Prickly Lettuce, Prickly Pear Cactus, Prickly Poppy, Priest's-crown, Prim, Primrose, Primula Veris, Prince's Feather, Prince's-pine, Privet, Prosopis pallida syn. Prosopis limensis, Provence Rose, Prunella, Prunella Vulgaris, Prunus Amygdalus, Prunus Dulcis, Prunus Serotina, Prunus Spinosa, Psidium Guajava, Psoralea, Psoralea corylifolia, Psoralea Fruit, Psyllium, Ptychopetalum ovata, Pueraria Lobata, Puff Ball, Pu gong ying, Pukeweed, Pu-kung-ying, Pulmonaria Officinalis, Pulsatilla, Pumpkin, Pumpkin Pine, Puncture Vine, Purging Buckthorn, Purple Angelica, Purple Betony, Purple Clover, Purple Coneflower, Purple Foxglove, Purple Leptandra, Purple Loosestrife, Purple Medic, Purple Passionflower, Purple Rocket, Purplestem Angelica, Purslane, Pygeum, Pygeum Africanum, Pyrethrum, Pyrola Umbellata, Quack Grass, Quaking Aspen, Quassia, Quassia Bark, Quassia Wood, Quebra Pedra, Queen Annes Lace, Queen Ofhe Meadow, Queensland Nut, Queen's Delight, Queen's-root, Quercus alba, Queue de Lezard, Quickbeam, Quick-set, Quince, Quinine Tree, Quinsy Berries, Quiverleaf, Race Ginger, Racoon Berry, Rainbowweed, Rashona, Raspberry, Raspberry Leaf, Rat's Tail, Rattlebush, Rattleroot, Rattlesnake Root, Rattleweed, Rau Ram, Rauwolfia, Rauwolfia Serpentina, Red Alder, Red Balm, Red Bearberry, Red-bearded, Red Bergamot, Redberry Tea, Red Bush Tea, Red Clover, Redcole, Red Dulse, Red Eyebright, Red Legs, Red Paint Root, Red Pollom, Red Poppy, Red Puccoon, Red Raspberry, Red Robin, Red Root, Red Root Sage, Red Rose, Red Sage, Red Sorrel, Red Sunflower, Red Tea, Red Trillium, Red-veined Dock, Reefer, Rehmannia, Rehmannia Glutinosa, Reishi, Rhamnus Cathartica, Rhamnus Frangula, Rhamnus Purshiana, Rheumatism Root, Rheumatism Weed, Rheum Palmatum, Rhodiola, Rhodiola sacra, Rhubarb, Rhus trilobata, Ribes Nigrum, Ribwort, Richweed, Ricinus Communis, Rimed scutatus, Ritha, Robinia Pseudoacacia, Rock Brake, Rock Fern, Rockweed, Roman Chamomile, Roman Cumin, Roman Fennel, Rooibos, Root Ofhe Holy Ghost, Roquette, Rosa Canina, Rosa Centifolia, Rosa Gallica, Rose, Rose Apple, Roselle, Rosemary, Rosemary Plant, Rose-noble, Rose Root, Rosin Rose, Rosmarinus Officinalis, Rosy Periwinkle, Rou Dou Kou, Round Buchu, Round-leaved Dock, Round-leafed Mint, Round-leaved Sorrel, Rowan Tree, Royal Herb, Royal Jasmine, Rub Cherry, Rubia tinctoria, Rubus Fructicosus, Rubus Idaeus, Rucola, Rue, Rugula, Rumara, Rumex Acetosella, Rumex Crispus, Rumex Hymenosepalus, Rumex Obtusifolius, Running Club Moss, Ruscus Aculeatus, Russian Chamomile, Russian Mustard, Rustic's Treacle, Ruta Graveolens, Sabal, Saccharum officinarum, Sacred Bark, Sacred Basil, Sacred Fig, Sacred Lotus, Sacred Plant, Sacred Sage, Sacred Tree, Sacred Water Lotus, Safflower, Saffron, Sagackhomi, Sage, Sage-leaved Germander, Sage Of Bethlehem, Saké, Salad Burnet, Salad Chervil, Salad Rocket, Salai Gugal, Salix Alba, Saloip, Salvia, Salvia apiana, Salvia hispanica, Salvia Miltiorrhiza, Salvia Officinalis, Salvia Sclarea, Sambucus Nigra, Sampson Root, Sandalwood, Sandberry, Sangre de Drago, Sangue de Drago, Sanguinaria, Sanguinaria Canadensis, Sanguisorba Minor, Sanguisorba Officinalis, Sanicle, Sanicula Europaea, Sanicula Marilandica, Santalum Album, Sapin, Sapindus mukorossi, Saponaria Officinalis, Sapote, Sarapia, Sarepta Mustard, Sarothamnus Scoparius, Sarpagandha, Sarsaparilla, Sassafras, Sassafras Albidum, Satan's Apple, Satavar, Satinflower, Satureja hortensis, Satureja montana, Saventaro, Savory, Saw Palmetto, Saxifrax, Scabish, Scabwort, Scarlet Bergamot, Scarlet Monarda, Scarlet Sage, Scarweed, Scented Fern, Scented Sumac, Schisandra, Schisandra Chinensis, Schizonepeta, Schizonepeta tenuifolia, Scopolia, Scopolia carniolica, Scotch Broom, Scotch Fir, Scotch Heather, Scotch Pine, Scots Pine, Scouring Rush, Scrofula Plant, Scrophularia Nodosa, Scurfy Pea, Scurvy Grass, Scurvy Weed, Scutellaria Baicalensis, Scutellaria Lateriflora, Sea Buckthorn, Sea Lettuce Flakes, Sea Oak, Sea Onion, Sea Parsley, Seaweed, Seawrack, Sedum Telephium, Self-Heal, Seneca Snakeroot, Senecio Aureus, Seneca Grass, Senega, Senega Root, Senna, Sereh, Serenoa Repens, Serpentary, Serpyllum, Setwall, Shameface, Shan-cha, Sha-ren, Shan-yao, Shatamull, Shatapushpa, Shatavari, Shave Grass, Shea Tree, Sheepberry, Sheep Sorrel, Sheng Di, Sheng Di Huang, Sheng Ti Huang, Shen Jiang, Shepherd's Knot, Shepherd's Needle, Shepherd's Purse, Shi Chang Pu, Shield Fern, Shih-lo, Shihuahuaco, Shiitake, Shiny Asparagus, Shirokikurage, Shoofly, Short Buchu, Short-leaved Buchu, Shovelweed, Shu Di Huang, Shu Ti Huang, Siberian Ginseng, Sicklewort, Silkweed, Silver Birch, Silver Fir, Silver Leaf, Silver-leaf Poplar, Silver Mint, Silver Pine, Silver Poplar, Silver Tree-ear Fungus, Silverweed, Silybum, Silymarin, Simply Jack, Sinapis alba, Skullcap, Skunkbush, Skunkbush Sumac, Skunk Cabbage, Slippery Elm, Sloe, Smallage, Smallanthus sonchifolius, Small Nasturtium, Smelling-stick, Smilax Uti Lis, Smooth Cicely, Smooth Strophanthus, Snakebite, Snake Lily, Snake Root, Snakeweed, Snapping Hazelnut, Snap-Wood, Snowball Tree, Snowdrop Tree, Snowflake, Snowflower, Snow Fungus, Soap Berry, Soapnut, Soapwort, Soft Pine, Solanum Dulcamara, Soldier's Cap, Soldier's Woundwort, Solidago Canadensis, Solidago Virgaurea, Solis Sponsa, Solomon's Seal, Solsequia, Son-before-the-father, Sorbus Aucuparia, Sour Dock, Sour Grass, Soursop, Sour Weed, Southern Ginseng, Southemwood, Sowberry, Soy, Soya, Soybean, Spanish Chamomile, Spanish Chestnut, Spanish Jasmine, Spanish Thyme, Spearmint, Speedwell, Spiceberry, Spicebush, Spicewood, Spicy Wintergreen, Spigelia Marilandica, Spike Lavender, Spiked Loosestrife, Spikenard, Spilanthes acmella, Spoonwood, Spoonwort, Spotted Alder, Spotted Thistle, Spring Cress, Spring Wintergreen, Square Stalk, Squawbush, Squaw Root, Squaw Tea, Squaw Vine, Squill, Stachys Officinalis, Stachys Palustris, Stagbush, Staghom, Stanchgrass, Star Anise, Starbloom, Star Flower, Star Fruit, Star Grass, Starweed, Starwort, Stellaria, Stellaria Media, Stickwort, St. Ignatius Bean, Stillingia Sylvatica, Stingnose, Stinking Benjamin, Stinking Christopher, Stinging Nettle, Stingless Nettle, Stinking Nightshade, Stinking Roger, Stinking Rose, Stinking Weed, Stinking Willie, Stinkweed, Stitchwort, St. John's Bread, St. John's Grass, St. John's Plant, St. John's Wort, St. Josephwort, Stonecrop, Stone Root, Strawberry, Stork's Bill, Strawberry Tomato, Strawberry Tree, Striped Alder, Strophanthus, Strophanthus Gratus, Strychnine, Strychnine Tree, Strychnos nux-vomica, Styrax Benzoin, Succory, Sudanese Tea, Sugar Cane, Sui Hoi, Suma, Su Nanesi, Sundew, Sunflower, Sunkfield, Sunthi, Surasa, Suterberry, Swallowwort, Swamp Cedar, Swamp Root, Sweating Plant, Sweet Almond, Sweet Balm, Sweet Basil, Sweet Bay, Sweet Birch, Sweet Bracken, Sweet Brake, Sweet Bugle, Sweet Cane, Sweet Chervil, Sweet Chestnut, Sweet Cicely, Sweet Clover, Sweet Coltsfoot, Sweet Cumin, Sweet Dock, Sweet Elm, Sweet Fennel, Sweet Fern, Sweet Flag, Sweet Flag Rhizome, Sweet Goldenrod, Sweet Grass, Sweet Iris, Sweet Lavender, Sweet Licorice, Sweet Lucerne, Sweet Majoram, Sweet Myrrh, Sweet Root, Sweet Rush, Sweet-scented Geranium, Sweet Tea Vine, Sweet Tongue, Sweet Violet, Sweetweed, Sweet Wood, Sweet Woodruff, Swertia, Swertia chirayita, Swine Snout, Symphytum Officinale, Symplocarpus Foetidus, Syrian Oregano, Syzygium cumini, Syzygium jambos, Tabebuia Spp., Tagara, Taheebo, Ta-huang, Tailed Cubebs, Tailed Pepper, Tailwort, Tall Nasturtium, Tallow Shrub, Tall Speedwell, Tall Veronica, Tamanu Nut Tree, Tamarind, Tamarindus Indica, Tamus, Tanacetum Parthenium, Tanacetum Vulgare, Tang Kuei, Tanners Bark, Tanner's-dock, Tan Shen, Tansy, Tap Aloe, Tapioca-root, Taraxacum Officinale, Tarragon, Tartar Root, Tarweed, Taxus Brevifolia, Teaberry, Teasel, Tea Tree, Te Limon, Telltime, Tenuifolia, Terminalia Aruna, Tetterberry, Tetterwort, Teucrium marum, Teucrium scorodonia, Thali, Theobroma Cacao, Thorn Apple, Thom Poppy, Thnomtree, Thorny Burr, Thoroughwort, Thousand-leaf, Thousand-seal, Three-leaved Caper, Three-leaved Nightshade, Throatwort, Thuja, Thuja Occidentalis, Thumb, Thunberg Fritillaria Bulb, Thyme, Thymus citriodorus, Thymus Serpyllum, Thymus Vulgaris, Tian Men Dong, Tibetan Rhodiola, Tien-shih-li, Ti Huang, Ti Huang Chiu, Tilia Americana, Tilia Cordata, Tilia Europea, Tipton Weed, Toad Flax, Tobacco Wood, Tokal, Tomillo, Tom Thumb Nasturtium, Tongue Grass, Tonka, Tonka Bean, Tonka Bean Tree, Tonquin Bean, Toothache Plant, Toothache Tree, Trachyspermum ammi, Treacle Mustard, Tree Moss, Tree of Joy, Tree of Life, Tree's Dandruff, Trefoil, Trembling Aspen, Trembling Poplar, Tremella fuciformis, Tribulus, Tribulus terrestris, Tricolor Garlic, Trifolium pratense, Trifolium repens, Trigonella, Trigonella Foenum-Graecum, Trillium, Trillium Erectum, Triticum aestivum, Tropaeolum minus, Tormentil, True Angostura, True Chamomile, True Lavender, True Oregano, True Sage, True Taragon, True Unicorn Root, Tse-lan, Tuber Root, Tuckahoe, Tulasi, Tulsi, Tumeric Root, Tuna Cactus, Turkey Burrseed, Turkish Oregano, Turmeric, Turnera Diffusa, Turtlebloom, Turtlehead, Tussilago Farfara, Twak, Twinflower, Tzu-mo-lo, Uassi, Ulmus Rubra, Uma, Umbrella Plant, Umcka, Umckaloabo, Una De Gato, Uncaria Tomentosa, Undaria pinnatifida, Upland Cotton, Upland Cress, Upland Cranberry, Uppagi, Upside-Down Tree, Urginea Maritima, Urtica Dioica, Usnea, Usnea spp., Uva Ursi, Vaccinium Macrocarpon, Vaccinium Myrtillus, Valerian, Valeriana Officinalis, Vandalroot, Vanilla, Vanilla fragrans, Vanilla Grass, Vanilla planifolia, Varuna, Vegetable Antimony, Vegetable Marrow, Vegetable Sulfur, Vegetable Tallow, Vegetable Wax, Venus' Basin, Venus'-hair Fern, Verbascum Thapsus, Verbena, Verbena Officinalis, Vermont Valerian, Vernonia Amygdalina, Veronica Beccabunga, Veronica Officinalis, Vervain, Vetiver, Vetiveria zizanioides, Vetivert, Viburnum Opulus, Viburnum Prunifolium, Vietnamese Cilantro, Vietnamese Coriander, Vietnamese Mint, Vinca Minor, Vinca Rosea, Vine, Viola Odorata, Viola Tricolor, Violet-bloom, Virginia Bugleweed, Virginia Dogwood, Virginia Prune, Virginia Skullcap, Virginia Snakeroot, Virginia Water Horehound, Viscum Album, Visnaga, Vitellaria paradoxa, Vitex, Vitex Agnus-Castus, Vitis Vinifera, Vomitroot, Wachsgagl, Wakame, Wake Robin, Waldmeister, Wallflower, Walnut, Wasabi, Wasabia japonica, Wasei, Water Bugle, Watercress, Water Dragon, Water Flag, Water Horehound, Water Hyssop, Water Lily, Water Mint, Water Pimpemel, Water Shamrock, Water Thistle, Wattle, Wax Cluster, Wax Dolls, Wax Myrtle, Way Bennet, Weeping Ash, Weeping Forsythia, Wheatgrass, Whinberry, Whippoorwill's-shoe, Whistling Thom, White Archangel, White Ash, White Birch, White Bird's-eye, White Bryony, White Cedar, White Ceremonial Sage, White Chamomile, White Clover, White Deadnettle, White Endive, White Flower De Luce, White Horehound, White Jelly-leaf, White Muer, White Lotus, White Mustard, White Nettle, White Pine, White Poplar, White Sage, White Tansy, Whitethorn, White Tree-ear, White Turmeric, White Walnut, White Willow, Whitten Tree, Whorlywort, Whortleberry, Whorts, Wild Allspice, Wild Angelica, Wild Black Cherry, Wild Brier, Wild Bryony, Wild Carrot, Wild Celery, Wild Chamomile, Wild Chicory, Wild Cotton, Wild Crocus, Wild Endive, Wild Fennel, Wild Geranium, Wild Hops, Wild Indigo, Wild Iris, Wild Lemon, Wild Lettuce, Wild Mandrake, Wild Marjoram, Wild Oats, Wild Opium, Wild Pansy, Wild Parsnip, Wild Passionflower, Wild Pieplant, Wild Rhubarb, Wild Rye, Wild Snakeroot, Wild Strawberries, Wild Succory, Wild Sunflower, Wild Sweetsop, Wild Tansy, Wild Teasel, Wild Thyme, Wild Tobacco, Wild Valerian, Wild Vine, Wild Yam, Willow, Willow Herb, Windflower, Wind Root, Wineberry, Winter Berry, Winterbloom, Winter Cherry, Winter Clover, Wintergreen, Winterlien, Winter Marjoram, Winter Savory, Winter Thyme, Winterweed, Witches'-moneybags, Witchgrass, Witch Hazel, Witch's Bells, Withania, Withania Somnifera, Wolfsbane, Wolf s Claw, Woman's Long Hair, Wood Betony, Woodbine, Wood Boneset, Wood Ear Fungus, Woodland Germander, Woodland Strawberry, Wood Licorice, Woodruff, Wood Sage, Wood Strawberry, Wood Turmeric, Wood Vine, Woodward, Woody Nightshade, Woolly Mint, Woolly Thistle, Worm Grass, Wormseed, Wormweed, Wormwood, Woundwort, Wu-pa-ho, Wu-wei-zi, Wycopy, Xiao-hue-xiang, Xi Shu, Xu Ku Cao, Yacon, Yacuma, Yang-Mei, Yape, Yarrow, Yasmin, Yasti Madhu, Yawroot, Yellow Bedstraw, Yellow Cedar, Yellow Dock, Yellow Eye, Yellow Ginseng, Yellow Indian Paint, Yellow Indian Shoe, Yellow Indigo, Yellow Jasmine, Yellow Jessamine, Yellow Lark's Heels, Yellow Locust, Yellow Loosestrife, Yellow Melilot, Yellow Mustard, Yellow Paint Root, Yellow Poppy, Yellow Puccoon, Yellow Rocket, Yellowroot, Yellow Thistle, Yellow Vine, Yerba, Yerba Buena, Yerba Mansa, Yerba Manza, Yerba Santa, Yin-hsing, Yin Yang Huo, Ylang Tree, Yohimbe, Yohimbine, Yucca, Yucca spp., Yueh-kuei, Yuma, Yu Mi Shu, Yuzu, Yuyu Chonta, Zaatar, Zacate Limon, Zanthoxylum Americanum, Zea Mays, Zedoary, or Zingiber Officinale.

Probiotics can also be included in cannabinoid compositions prepared according to the invention. Examples of suitable probiotics include, but are not limited to, Acinetobacter calcoaceticus, Arthrobacter agilis, Arthrobacter citreus, Arthrobacter globiformis, Arthrobacter luteus, Arthrobacter simplex, Azotobacter chroococcum, Azotobacter paspali, Azospirillum brasiliense, Azospirillum lipoferum, Bacillus ssp. (e.g., Bacillus brevis, Bacillus coagulans, Bacillus laterosporus, Bacillus marcerans, Bacillus pumilus, Bacillus polymyxa, Bacillus sphaericus, Bacillus subtilis), Bacteroides lipolyticum, Bacteroides succinogenes, Bifidobacterium ssp. (e.g., Bifidobacterium animalis lactis, Bifidobacterium bifidum, Bifidobacterium infantis, Bifidobacterium lactis, Bifidobacterium longum, Bifidobacterium animalis, Bifidobacterium breve), Brevibacterium lipolyticum, Brevibacterium stationis, Enterococcus faecium, Kurthia zopfii, Lactobacillus ssp. (e.g., Lactobacillus acidophilus, Lactobacillus brevis, Lactobacillus bulgaricus, Lactobacillus casei, Lactobacillus delbrueckii LE, Lactobacillus gasseri, Lactobacillus helveticus, Lactobacillus lacris, Lactobacillus paracasei, Lactobacillus plantarumtarum, Lactobacillus reuteri, Lactobacillus rhamnosus, Lactobacillus salivarius, Lactobacillus sporogenes), Myrothecium verrucaria, Pseudomonas calcis, Pseudomonas dentrificans, Pseudomonas fluorescens, Pseudomonas glathei, Phanerochaete chrysosporium, Saccharomyces boulardii, Saccharomyces cerevisiae, Streptococcus thermophilus, Streptomyces fradiae, Streptomyces cellulosae, Streptomyces griseoflavus, and combinations thereof.

Homeopathic remedies can also be included in cannabinoid compositions prepared according to the invention. Examples of suitable homeopathic remedies and indications that can be treated with homeopathic remedies include, but are not limited to, adrenocorticotropic hormone (ATCH), Abies Canadensis, Abies Nigra, Abrotanum, Absinthium, Acacia Arabica, Acalypha Indica, Acetaldehyde, Acetanilidum, Aceticum Acidum, Acetylsalicylicum Acidum, Achyranthis Calea, Aconite or Aconitum Nap, Aconitum Ferox, Aconitum Lycoctonum, Aconitum Napellus, Aconitum, Radix, Acorus Calamus or Calamus, Actaea Spicata Acrylate, Actaea Rac or Cimicifuga, Actaea Spic, Adamas, Adelheidsquelle, Adenosinum Cyclophosphoricum, Adeps Suillus, Adipose Tissue, Adonis Vernalis, Adrenal Cortex, Adrenal Gland, Adrenalinum or Epinephrine, Adrenocorticotrophin, Aesculinum, Aesculus Camea, Flos, Aesculus Glabra, Aesculus Hippocastanum, Aesculus Hippocastanum Flos, Aethiops Antimonialis, Aethiops Mercurialis-Mineralis, Aethusa Cynapium, Agaricinum, Agaricus Campanulatus, Agaricus Campestris, Agaricus Citrinus, Agaricus Emeticus, Agaricus Muscarius, Agaricus Pantherinus, Agaricus Phalloides, Agaricus Procerus, Agaricus Semiglobatus, Agaricus Stercorarius, Agave Americana, Agave Tequilana, Agnus Castus, Agraphis Nutans, Agrimonia Eupatoria, Agrimonia Eupatoria, Flos, Agrimonia Odorata, Flos, Agrostemma Githago, Ailanthus Glandulosus, Aletris Farinosa, Alfalfa, Alisma Plantago, Allium Cepa, Allium Sativum, Alloxanum, Alnus Glutinosa, Alnus Serrulata, Aloe Socotrina, Alstonia Constricta, Alstonia Scholaris, Althaea Officinalis, Alumen or Alum, Alumina, Alumina Silicata, Aluminum Metallicum, Aluminum Muriaticum, Ambra Grisea, Ambrosia Artemisiaefolia, Ammi Visnaga, Ammoniacum Gummi, Ammonium Aceticum, Ammonium Benzoicum, Ammonium Bromatum, Ammonium Carbonicum, Ammonium Causticum, Ammonium Citricum, Ammonium Iodatum, Ammonium Muriaticum, Ammonium Nitricum, Ammonium Phosphoricum, Ammonium Picricum, Ammonium Tartaricum, Ammonium Valerianicum, Ammonium Vanadium, Amorphophallus Rivieri, Ampelopsis Quinquefolia, Amygdala Amara, Amygdalae Amarae Aqua, Amygdalae Amarae Oleum, Amygdalus Persica, Amyl Nitrosum, Anacardium Occidentale, Anacardium Orientate, Anagallis Arvensis, Ananassa, Anas Barbariae Hepatis Et Cordis Extractum, Anatherum Muricatum, Anchusa Officinalis, Anemone Nemorosa, Anemopsis Californica, Anethum Graveolens, Angelica Archangelica, Angelica Atropurpurea, Angelica Sinensis, Radix, Angophora Lanceolata, Angustura Vera, Anhalonium Lewinii, Anilinum, Anilinum Sulphuricum, Anisum, Anthemis Nobilis, Anthemis Pyrethrum, Anthoxanthum Odoratum, Anthracinum (anthrax), Antimonium Arsenicicum, Antimonium Crudum, Antimonium Iodatum, Antimonium Muriaticum, Antimonium Oxydatum, Antimonium Sulphuratum Aureum, Antimonium Tartaricum, Antipyrinum, Apatite, Apiolum, Apis Mellifica, Apis Venenum Purum, Apium Graveolens, Apocynum Androsaemifolium, Apocynum Cannabinum, Apomorphinum, Apomorphinum Muriaticum, Aqua Marina, Aquilegia Vulgaris, Aralia Hispida, Aralia Quinquefolia, Aralia Racemosa, Aranea Diadema, Arbutinum, Arbutus Andrachne, Areca Catechu, Argemone Mexicana, Argentum Cyanatum, Argentum Iodatum, Argentum Metallicum, Argentum Muriaticum, Argentum Nitricum, Argentum Oxydatum, Argentum Phosphoricum, Aristolochia Clematitis, Aristolochia Milhomens, Aristolochia Serpentaria, Amica Montana, Amica Montana, Radix, Arsenicum Album, Arsenicum Bromatum, Arsenicum Iodatum, Arsenicum Metallicum, Arsenicum Sulphuratum Flavum, Arsenicum Sulphuratum Rubrum, Artemisia Vulgaris, Arum Dracontium, Arum Italicum, Arum Maculatum, Arum Triphyllum, Arundo Mauritanica, Asafoetida, Asarum Canadense, Asarum Europaeum, Asclepias Curassavica, Asclepias Incarnata, Asclepias Syriaca, Asclepias Tuberosa, Asclepias Vincetoxicum, Asclepias Vincetoxicum Folia, Asimina Triloba, Asparagus Officinalis, Asperula Odorata, Astacus Fluviatilis, Asterias Rubens, Astragalus Menziesii, Atropinum, Atropinum Sulphuricum, Aurum Bromatum, Aurum Iodatum, Aurum Met, Arum Mur, Aurum Muriaticum Kalinatum, Aurum Muriaticum Natronatum, Aurum Sulphuratum, Avena Sativa, Aviaire, Azadirachta Indica, Bacillinum of Burnet, Badiaga, Baja, Balsamum Peru, Baptisia Tinctoria, Barosma Cren, Baryta Acetica, Baryta Carbonica, Baryta Iodata, Baryta Muriatica, BCG, Belladonna, Belladonna, Radix, Bellis Perennis, Benzinum, Benzinum Dinitricum, Benzoicum Acidum, Benzoinum, Berberinum, Berberis Aquifolium, Berberis Vulgaris, Berberis Vulgaris, Fructus, Beryllium Metallicum, Beta Vulgaris, Betainum Muriaticum, Betula Pendula, Cortex, Betula Pendula, Folia, Bismuthum Metallicum, Bismuthum Oxydatum, Bismuthum Subnitricum, Bixa Oreliana, Blatta Americana, Blatta Orientalis, Boldo, Boletus Luridus, Boletus Satanas, Bombyx Processionea, Borago Officinalis, Borax, Boricum Acidum, Botulinum, Bovista, Brassica Napus, Bromelain, Bromium, Bromus Ramosus, Flos, Brucinum, Bryonia Alba, Bufo Rana, Bunias Orientalis, Buthus Australis, Butyricum Acidum, Buxus Sempervirens, Cacao, Cactus Grandiflorus, Cadmium Bromatum, Cadmium Iodatum, Cadmium Metallicum, Cadmium Muriaticum, Cadmium Sulphuratum, Cadmium Sulphuricum, Caffeinum, Cahinca, Cajuputum, Caladium Seguinum, Calcarea Acetica, Calcarea Arsenicica, Calcarea Carbonica, Calcarea Caustica, Calcarea Fluorica, Calcarea Hypochlorata, Calcarea Hypophosphorosa, Calcarea Iodata, Calcarea Lactica, Calcarea Muriatica, Calcarea Oxalica, Calcarea Phosphorica, Calcarea Picrata, Calcarea Silicata, Calcarea Sulphurica, Calendula Officinalis, Calluna Vulgaris, Flos, Calotropis Gigantea, Caltha Palustris, Camphora, Camphora Monobromata, Camphoricum Acidum, Canchalagua, Candida Albicans, Candida Parapsilosis, Canine Dapp, Cantharidinum, Cantharis, Capsicum, Capsicum Annuum, Carbo Animalis, Carbo Vegetabilis, Carbolicum Acidum, Carboneum, Carboneum Chloratum, Carboneum Hydrogenisatum, Carboneum Oxygenisatum, Carboneum Sulphuratum, Carcinosinum, Cardiospermum, Carduus Benedictus, Carduus Marianus, Carpinus Betulus, Flos, Cartilago Suis, Carum Carvi, Cascarilla, Cassada, Castanea Sativa, Flos, Castanea Vesca, Castor Equi, Castoreum, Catalpa Bignonioides, Caulophyllum Thalictroides, Causticum, Ceanothus Americanus, Cedron, Celtis Occidentalis, Cenchris Contortrix, Centaurea Tagana, Centaurium Umbellatum, Flos, Cephalanthus Occidentalis, Cerasus Virginiana, Ceratostigma Willmottianum, Flos, Cereus Bonplandii, Cereus Serpentinus, Cerium Oxalicum, Cetraria Islandica, Chamomilla, Cheiranthus Cheiri, Chelidonium Majus, Chelidonium Majus, Radix, Chelone Glabra, Chenopodii Glauci Aphis, Chenopodium Anthelminticum, Chenopodium Vulvaria, Chimaphila Maculata, Chimaphila Umbellata, Chininum Arsenicicum, Chininum Arsenicosum, Chininum Muriaticum, Chininum Purum, Chininum Salicylicum, Chininum Sulphuricum, Chionanthus Virginica, Chloralum, Chloramphenicolum, Chlorinum, Chloroforum, Chlorpromazinum, Cholera, Cholesterinum, Cholinum, Chromicum Acidum, Chromium Kali Sulphuricum, Chromium Oxydatum, Chromium Sulphuricum, Chrysanthemum Leucanthemum, Chrysarobinum, Cicer Arietinum, Cichorium Intybus, Cichorium Intybus, Flos, Cicuta Maculata, Cicuta Virosa, Cimex Lectularius, Cimicifuga Racemosa, Cina, Cinchona Officinalis, Cinchonium Sulphuricum, Cineraria Maritima, Cineraria Maritima, Succus, Cinnamomum, Cistus Canadensis, Citricum Acidum, Citrus Decumana, Citrus Limonum, Citrus Vulgaris, Clematis Erecta, Clematis Virginiana, Clematis Vitalba, Flos, Clematis Vitalba, Folia, Cobaltum Metallicum, Cobaltum Muriaticum, Cobaltum Nitricum, Coccinella Septempunctata, Cocculus Indicus, Coccus Cacti, Cochlearia Armoracia, Cochlearia Officinalis, Coenzyme A, Coffea Cruda, Coffea Tosta, Colchicinum, Colchicum Autumnale, Colibacillinum, Collinsonia Canadensis, Colocynthinum, Colocynthis, Colostrum, Comocladia Dentata, Conchiolinum, Condurango, Coniinum, Coniinum Bromatum, Conium Maculatum, Convallaria Majalis, Convolvulus Arvensis, Copaiva Officinalis, Corailium Rubrum, Corallorhiza Odontorhiza, Coriaria Ruscifolia, Comus Altemifolia, Comus Circinata, Cornus Florida, Cortisone Aceticum, Corydalis Canadensis, Cotyledon Umbilicus, Coumarinum, Crataegus Oxyacantha, Cresolum, Crocus Sativus, Crotalus Cascavella, Crotalus Horridus, Croton Tiglium, Crotonchloralum, Cubeba Officinalis, Cucurbita Citrullus, Cucurbita Pepo. Flos, Cucurbita Pepo, Semen, Culex Musca, Cuphea Petiolata, Cupressus Australis, Cupressus Lawsoniana, Cuprum Aceticum, Cuprum Ammonio-Sulphuricum, Cuprum Arsenicosum, Cuprum Carbonicum, Cuprum Metallicum, Cuprum Muriaticum, Cuprum Nitricum, Cuprum Oxydatum Nigrum, Cuprum Sulphuricum, Curare, Cyclamen Europaeum, Cydonia Vulgaris, Cynara Scolymus, Cynodon Dactylon, Cypripedium Pubescens, Cysteinum, Cytisus Scoparius, Damiana, Daphne Indica, Datura Arborea, Datura Metel, DDT, Delphininum, Derris Pinnata, Dichapetalum, Dictamnus Albus, Digitalinum, Digitalis Purpurea, Digitoxinum, Dioscorea Villosa, Dioscoreinum, Diphtherinum, Diphtherotozinum, Diptherinum, Diptherotoxinum, Dirca Palustris, DNA, Dolichos Pruriens, Doryphora Decemlineata, Draba Vema, Drosera Rotundifolia, DTTAB (Diptheria), Duboisia Myoporoides, Dulcamara, Dulcamara, Flos, Dysentery, E. Coli, Ear, Labyrinth of (inner ear), Ear, Middle, Eberthinum, Echinacea Angustifolia, Echinacea Purpurea, Elaeis Guineensis, Elaps Corallinus, Elaterium, Embryo Suis, Emetinum, Enterotoccinum, Eosinum Natrum, Ephedra Vulgaris, Epigaea Repens, Epilobium Palustre, Epiphegus Virginiana, Equisetum Arvense, Equisetum Hyemale, Eranthis Hyemalis, Erechtites Hieracifolia, Erigeron Canadensis, Eriodictyon Califomicum, Erodium, Eryngium Aquaticum, Eryngium Maritimum, Erythraea Centaurium, Eschscholtzia Califomica, Eserinum, Etherum, Ethylicum, Ethylum Nitricum, Eucalyptol, Eucalyptus Globulus, Eugenia Caryophyllata, Eugenia Jambosa, Euonymus Atropurpureus, Euonymus Europaeus, Eupatorium Aromaticum, Eupatorium Cannabinum, Eupatorium Perfoliatum, Eupatorium Purpureum, Euphorbia Amygdaloides, Euphorbia Corollata, Euphorbia Cyparissias, Euphorbia Hypericifolia, Euphorbia Lathyris, Euphorbia Pilulifera, Euphorbium Officinarum, Euphrasia Officinalis, Eupion, Eyebright herb, Fagopyrum Esculentum, Fagus Sylvatica, Fagus Sylvatica, Flos, Fel Tauri, Ferrum Aceticum, Ferrum Arsenicicum, Ferrum Bromatum, Ferrum Carbonicum, Ferrum Citricum, Ferrum Cyanatum, Ferrum Iodatum, Ferrum Lacticum, Ferrum Metallicum, Ferrum Muriaticum, Ferrum Pernitricum, Ferrum Phosphoricum, Ferrum Picricum, Ferrum Sulphuricum, Ferrum Tartaricum, Ferula Glauca, Ficus Religiosa, Filix Mas, Foeniculum Vulgare, Folliculinum, Formalinum, Formica Rufa, Formicum Acidum, Fragaria Vesca, Franciscea Uniflora, Fraxinus Americana, Fraxinus Excelsior, Fuchsinum, Fucus Vesiculosus, Fumaria Officinalis, Fumaricum Acidum, Funiculus Umbilicalis Suis, Galanthus Nivalis, Galega Officinalis, Galium Aparine, Gallicum Acidum, Galphimia Glauca, Gambogia, Garlic, Gaultheria Procumbens, Gelsemium Sempervirens, Genista Tinctoria, Gentiana Cruciata, Gentiana Lutea, Gentiana Quinqueflora, Gentianella Amarella, Flos, Geranium Maculatum, Geranium Robertianum, Geum Rivale, Geum Urbanum, Ginkgo Biloba, Glandula Suprarenalis Suis, Glechoma Hederacea, Glonoinum, Glycerinum, Glycogenum, Glycyrrhiza Glabra, Gnaphalium Leontopodium, Gnaphalium Polycephalum, Gnaphalium Uliginosum, Gonotoxinum, Gossypium Herbaceum, Granatum, Graphites, Gratiola Officinalis, Grindelia, Guaco, Guaiacum, Guarea Trichilioides, Guatteria Gaumeri, Gunpowder, Gymnocladus Canadensis, Haematoxylon Campechianum, Haemophilus Infl. B, Hair Bulb, Pilo Sebaceous Zone, HamamelisVirginiana, Haronga Madagas-cariensis, Hedeoma Pulegioides, Hedera Helix, Hekla Lava, Helianthemum Nummularium, Flos, Helianthus Annuus, Heliotropium Peruvianum, Helix Tosta, Helleborus Foetidus, Helleborus Niger, Helleborus Viridis, Heloderma, Helonias Dioica, Hepar Suis, Hepar Sulphuris Calcareum, Hepar Sulphuris Kalinum, Hepatica Triloba, Hepatitis A, Hepatitis B, Hepatitis C, Heracleum Sphondylium, Herpes Zoster, Hippozaeninum, Hippuricum Acidum, Hirudinum, Histaminum Hydrochloricum, Hoang-Nan, Hoitzia Coccinea, Holarrhena Antidysenterica, Homarus, Hottonia Palustris, Flos, Humulus Lupulus, Hum Brasiliensis, Hum Crepitans, Hydrangea Arborescens, Hydrastininum Muriaticum, Hydrastis Canadensis, Hydrocotyle Asiatica, Hydrocyanicum Acidum, Hydrofluoricum Acidum, Hydrophis Cyanocinctus, Hydrophyllum Virginianum, Hyoscyaminum, Hyoscyaminum, Hydrobromatum, Hyoscyamus Niger, Hypericum Perforatum, Hypothalamus, Iberis Amara, Ichthyolum, Ignatia Amara, Ilex Aquifolium, Ilex Aquifolium, Flos, Ilex Paraguariensis, Illicium Anisatum, Impatiens Glandulifera, Flos, Imperatoria Ostruthium, Indigo, Indium Metallicum, Indolum, Influenzinum, Inula Helenium, lodium, lodoformum, Ipecacuanha, Ipomoea Stans, Iridium Metallicum, Iris Florentina, Iris Foetidissima, Iris Germanica, Iris Tenax, Iris Versicolor, Jacaranda Caroba, Jalapa, Jasminum Officinale, Jasper, Jatropha Curcas, Jatropha Urens, Jequirity, Jonesia Asoca, Juglans Cinerea, Juglans Regia, Juglans Regia, Flos, Juncus Effusus, Juniperus Communis, Juniperus Virginiana, Justicia Adhatoda, Kali Aceticum, Kali Arsenicosum, Kali Bichromicum, Kali Bromatum, Kali Carbonicum, Kali Causticum, Kali Chloricum, Kali Chromicum, Kali Cyanatum, Kali Ferrocyanatum, Kali lodatum, Kali Muriaticum, Kali Nitricum, Kali Oxalicum, Kali Permanganicum, Kali Phosphoricum, Kali Picricum, Kali Silicatum, Kali Sulphuricum, Kali Tartaricum, Kali Telluricum, Kalmia Latifolia, Kamala, Karaka, Karwinskia Humboldtiana, Kino Australiensis, Kousso, Kreosotum, Labumum Anagyroides, Lac Caninum, Lac Defloratum, Lac Felinum, Lac Matemum, Lac Vaccinum, Lacerta Agilis, Lachesis Mutus, Lachnanthes Tinctoria, Lacticum Acidum, Lactuca Virosa, Lamium Album, Lapis Albus, Lappa Major, Larix Decidua, Flos, Lathyrus Cicera, Lathyrus Sativus, Latrodectus Katipo, Latrodectus Mactans, Laurocerasus, Lecithin granules, Lecithin potenized, Ledum Palustre, Lemna Minor, Leonurus Cardiaca, Lepidium Bonariense, Leptandra Virginica, Lespedeza Capitata, Levico, Levisticum Officinale, Levomepromazinum, Liatris Spicata, Lilium Tigrinum, Limulus, Linaria Vulgaris, Linum Catharticum, Linum Usitatissimum, Lithium Benzoicum, Lithium Bromatum, Lithium Carbonicum, Lithium Muriaticum, Lobelia Cardinalis, Lobelia Erinus, Lobelia Inflata, Lobelia Purpurescens, Lobelia Syphilitica, Lobelinum, Lolium Temulentum, Lonicera Caprifolium, Flos, Lonicera Periclymenum, Lonicera Xylosteum, Lophophytum Leandri, Luesinum, Luffa Operculata, Lupulinum, Lycopersicum Esculentum, Lycopodium Clavatum, Lycopus Virginicus, Lysimachia Nummularia, Lyssin, Lyssinum, Macrotinum, Magnesia Carbonica, Magnesia Muriatica, Magnesia Oxydata, Magnesia Phosphorica, Magnesia Sulphurica, Magnesium Metallicum, Magnolia Glauca, Magnolia Grandiflora, Malaria Off, Malus Pumila, Flos, Mancinella, Mandragora Officinarum, Manganum Aceticum, Manganum Carbonicum, Manganum Metallicum, Manganum Muriaticum, Manganum Oxydatum Nativum, Manganum Oxydatum Nigrum, Manganum Phosphoricum, Manganum Sulphuricum, Mangifera Indica, Marrubium Vulgare, Matico, Matthiola Graeca, Medorrhinum (Gonorrheal virus), Medulla Ossis Suis, Medusa, Melastoma Ackermani, Melilotus Alba, Melilotus Officinalis, Melissa Officinalis, Menispermum Canadense, Mentha Piperita, Mentha Pulegium, Mentha Viridis, Mentholum, Menyanthes Trifoliata, Mephitis Mephitica, Mercurialis Perennis, Mercurius Aceticus, Mercurius Auratus, Mercurius Bromatus, Mercurius Corrosivus, Mercurius Cum Kali Iodatus, Mercurius Cyanatus, Mercurius Dulcis, Mercurius Iodatus Flavus, Mercurius Iodatus Ruber, Mercurius Methylenus, Mercurius Nitricus, Mercurius Praecipitatus Albus, Mercurius Praecipitatus Ruber, Mercurius Solubilis, Mercurius Sulphocyanatus, Mercurius Sulphuratus Ruber, Mercurius Sulphuricus, Mercurius Vivus, Methylene Blue, Mezereum, Millefolium, Mimosa Pudica, Mimulus Guttatus, Flos, Mitchella Repens, Momordica Balsamina, Mononucleosis, Monotropa Uniflora, Morbillinum (Measles), Moschus, Mucosa Nasalis Suis, Mullein Essence, Murex Purpurea, Muriaticum Acidum, Musa Sapientum, Mygale, Myosotis Arvensis, Myrica Cerifera, Myristica Sebifera, Myrrha, Myrtus Communis, Nabalus Serpentarius, Nadidum, Naja Tripudians, Naphthalinum, Narceinum, Narcissus, Pseudo-, Narcissus, Narcotinum, Nasturtium Aquaticum, Natrum Arsenicicum, Natrum Bicarbonicum, Natrum Bromatum, Natrum Carbonicum, Natrum Fluoratum, Natrum Hypochlorosum, Natrum Lacticum, Natrum Muriaticum, Natrum Nitricum, Natrum Nitrosum, Natrum Oxalaceticum, Natrum Phosphoricum, Natrum Pyruvicum, Natrum Salicylicum, Natrum Silicofluoricum, Natrum Sulphuratum, Natrum Sulphuricum, Natrum Sulphurosum, Negundo, Nepenthes, Nepeta Cataria, Niccolum Carbonicum, Niccolum Metallicum, Niccolum Sulphuricum, Nicotinamidum, Nicotinum, Nitri Spiritus Dulcis, Nitricum Acidum, Nitrogenum Oxygenatum, Nitromuriaticum Acidum, Nosode Kit, Nosode-Select your own, Nuclear Radiation, Nuphar Luteum, Nux Moschata, Nux Vomica, Nymphaea Odorata, Ocimum Basilicum, Ocimum Canum, Ocimum Sanctum, Oenanthe Crocata, Oenothera Biennis, Olea Europaea, Flos, Oleander, Oleum Animale, Oleum Carvi, Oleum Morrhuae, Oleum Ricini, Oleum Santali, Olibanum, Oniscus, Ononis Spinosa, Onopordum, Onosmodium Virginianum, Oophorinum, Opuntia Vulgaris, Orchitinum, Oreodaphne Califomica, Origanum Majorana, Omithogalum Umbellatum, Omithogalum Umbellatum, Flos, Oroticum Acidum, Oscillococcinum, Osmium Metallicum, Ostrya, Ova Tosta, Ovi Gallinae Pellicula, Oxalicum Acidum, Oxalis Acetosella, Oxydendrum Arboreum, Oxytropis Lambertii, Paeonia Officinalis, Palladium Metallicum, Paloondo, Pancreas Suis, Pancreatinum, Paraffinum, Parathormonum, Parathyroid, Paratyphoidinum B, Pareira Brava, Parietaria Officinalis, Paris Quadrifolia, Paronichia Illecebrum, Parotidinum(Mumps), Parthenium, Passiflora Incanata, Pastinaca Sativa, Paullinia Pinnata, Paullinia Sorbilis, Pecten, Pediculus Capitis, Penicillinum, Penthorum Sedoides, Pepsinum, Perhexilinum, Persea Americana, Pertussinum (Whooping Cough), Petiveria Tetrandra, Petroleum, Petroselinum Sativum, Phallus Impudicus, Phaseolus, Phellandrium Aquaticum, Phenacetinum, Phenobarbitalum, Phloridzinum, Phosphoricum Acidum, Phosphorus, Physalis Alkekenge, Physotigma Venenosum, Phytolacca Decandra, Pichi, Picricum Acidum, Picrotoxinum, Pilocarpinum, Pilocarpinum Muriaticum, Pilocarpinum Nitricum, Pilocarpus, Pimenta Officinalis, Pimpinella Saxifraga, Pinus Lambertiana, Pinus Sylvestris, Pinus Sylvestris, Flos, Piper Methysticum, Piper Nigrum, Piperazinum, Piscidia Erythrina, Pituitarum Posterium, Pix Liquida, Placenta Totalis Suis, Plague, Plantago Major, Platanus, Platinum Metallicum, Platinum Muriaticum, Plectranthus Fruticosus, Plumbago Littoralis, Plumbum Aceticum, Plumbum Carbonicum, Plumbum Chromicum, Plumbum Iodatum, Plumbum Metallicum, Pneumococcinum, Podophyllinum, Podophyllum Peltatum, Polio, Polygonum Punctatum, Polygonum Sagittatum, Polyporus Officinalis, Polyporus Pinicola, Populus Candicans, Populus Tremula, Flos, Populus Tremuloides, Potentilla Anserina, Pothos Foetidus, Primula Obconica, Primula Veris, Primula Vulgaris, Proteus Bulgaris, Proteus Vulgaris, Prunus Cerasifera, Flos, Prunus Padus, Prunus Spinosa, Prunus Virginiana, Psorinum, Ptelea Trifoliata, Pulex Irritans, Pulsatilla Niger, Pulsatilla Nuttalliana, Pyrethrum Parthenium, Pyridoxinum Hydrochloricum, Pyrogenium-sepsis, Pyrus Americana, Quassia Amara, Quebracho, Quercus Glandium Spiritus, Quercus Robur, Quercus Robur, Flos, Quillaja Saponaria, Radium Bromatum, Ranunculus Acris, Ranunculus Bulbosus, Ranunculus Ficaria, Ranunculus Glacialis, Ranunculus Repens, Ranunculus Sceleratus, Raphanus Sativus, Ratanhia, Rauwolfia Serpentina, Reserpinum, Resina Laricis, Resorcinum, Rhamnus Califomica, Rhamnus Cathartica, Rhamnus Frangula, Rhamnus Purshiana, Rheum Officinale, Rhodium Metallicum, Rhododendron Chrysanthum, Rhus Aromatica, Rhus Diversiloba, Rhus Glabra, Rhus Toxicodendron, Rhus Venenata, Riboflavinum, Ricinus Communis, RNA, Robinia Pseudoacacia, Rock Water, Rosa Canina, Rosa Canina, Flos, Rosa Damascena, Rosmarinus Officinalis, Rubella (German Measles), Rubeola (Measles), Rubia Tinctorum, Rumex Acetosa, Rumex Crispus, Rumex Obtusifolius, Russula Foetens, Ruta Graveolens, Sabidilla, Sabal Serrulata, Sabina, Saccharinum, Saccharum Lactis, Saccharum Officinale, Salicinum, Salicylicum Acidum, Salix Alba, Salix Nigra, Salix Purpurea, Salix Vitellina, Flos, Salmonella, Salol, Salvia Officinalis, Samarskite, Sambucus Canadensis, Sambucus Nigra, Sanguinaria Canadensis, Sanguinarinum Nitricum, Sanicula, Santoninum, Saponaria Officinalis, Saponinum, Sarcode-Select your own organ remedy, Sarcolacticum Acidum, Sarracenia Purpurea, Sarsaparilla, Sassafras Officinale, Scammonium, Scarlatinum, Secale-Ergot Schinus Molle, Scilla Maritima, Scleranthus Annuus, Flos, Scolopendra, Scolopendrium Vulgare, Scopolaminum Hydrobromidum, Scrophularia Nodosa, Scutellaria Lateriflora, Secale Comutum, Secale-Ergot, Sedum Acre, Selenium Metallicum, Sempervivum Tectorum, Senecio Aureus, Senecio Jacobaea, Senega Officinalis, Senna, Sepia, Serum Anguillae, Serum Anticolibacillaire, Serum De Yersin, Serum Equi, Shigella, Silica Marina, Silicea, Silphium Laciniatum, Sinapis Alba, Sinapis Arvensis, Flos, Sinapis Nigra, Sinusitisinum, Sium Latifolium, Skatolum, Skookum Chuck, Slag, Solaninum, Solanum Arrebenta, Solanum Carolinense, Solanum Mammosum, Solanum Nigrum, Solanum Oleraceum, Solanum Tuberosum, Solidago Virgaurea, Sparteinum Sulphuricum, Spigelia Anthelmia, Spigelia Marilandica, Spilanthes Oleracea, Spinacia, Spiraea Ulmaria, Spiranthes Autumnalis, Spongia Encephalitis, Spongia Tosta, Stachys Betonica, Stannum Iodatum, Stannum Metallicum, Staphyloccoccus Aureus, Staphylococcinum, Staphylotoxinum, Staphysagria, Stellaria Media, Sterculia Acuminata, Stibium Metallicum, Sticta Pulmonaria, Stigmata Maidis, Stillingia Sylvatica, Stramonium, Streptococcinum, Strontium Bromatum, Strontium Carbonicum, Strontium Nitricum, Strophanthus Hispidus, Strophanthus Sarmentosus, Strychninum, Strychinum Arsenicicum, Strychinum Nitricum, Strychninum Phosphoricum, Strychninum Sulphuricum, Succinicum Acidum, Succinum, Sulphanilamidum, Sulphonalum, Sulphur, Sulphur Hydrogenisatum, Sulphur Iodatum, Sulphuricum Acidum, Sulphurosum Acidum, Sumbul, Symphoricarpus Racemosus, Symphytum Officinale, Syphilinum (Luesinum), Syzygium Jambolanum, Tabacum, Tamus Communis, Tanacetum Vulgare, Tanghinia Venenifera, Tannicum Acidum, Taraxacum Officinale, Taraxacum Officinale, Radix, Tarentula Cubensis, Tarentula Hispana, Tartaricum Acidum, Taxus Baccata, Tellurium Metallicum, Teplitz, Terebinthina, Tetanotoxinum, Tetradymite, Teucrium Marum, Teucrium Scorodonia, Thallium Metallicum, Thaspium Aureum, Thea Sinensis, Theobrominum, Theridion, Thiaminum Hydrochloricum, Thioproperazinum, Thiosinaminum, Thlaspi Bursa-Pastoris, Thuja Lobbi, Thuja Occidentalis, Thymolum, Thymus Serpyllum, Thyroidinum, Tilia Europaea, Titanium Metallicum, Tongo, Tormentilla, Torula Cerevisiae, Toxicophis Pugnax, Tradescantia Diuretica, Tribulus Terrestris, Trifolium Pratense, Trifolium Repens, Trillium Pendulum, Trimethylaminum, Triosteum Perfoliatum, Triticum Repens, Tropaeolum Majus, Tuberculinum, Tuberculinum Residuum, Tussilago Farfara, Tussilago Fragrans, Tussilago Petasites, Typhoidinum, Ulex Europaeus, Flos, Ulmus Fulva, Ulmus Procera, Flos, Upas Tieute, Uranium Nitricum, Urea, Uricum Acidum, Urtica Crenulata, Urtica Dioica, Urtica Urens, Usnea Barbata, Ustilago Maidis, Uva-Ursi herb, Uva-Ursi, V.A.B.-BCG, Vaccinium Myrtillus, Vaccinotoxinum, Valeriana Officinalis, Vanadium Metallicum, Varicella enus Mercenaria (Chicken Pox), Variolinum (Smallpox), Veratrinum, Veratrum Album, Veratrum Nigrum, Veratrum Viride, Verbascum Thapsus, Verbena Hastata, Verbena Officinalis, Verbena Officinalis, Flos, Veronica Beccabunga, Veronica Officinalis, Vesicaria, Vespa Crabro, Viburnum Opulus, Viburnum Prunifolium, Vinca Minor, Viloa Odorata, Viola Tricolor, Vipera Berus, Viscum Album, Vitamin B12, Vitamin K, Vitis Vinifera, Flos, Wiesbaden, Wyethia Helenioides, X-Ray, Xanthoxylum Fraxineum, Xerophyllum Asphodeloides, Yohimbinum, Yucca Filamentosa, Zincum Aceticum, Zincum Bromatum, Zincum Carbonicum, Zincum Cyanatum, Zincum Gluconicum, Zincum Iodatum, Zincum Metallicum, Zincum Muriaticum, Zincum Oxydatum, Zincum Phosphoratum, Zincum Picricum, Zincum Sulphuricum, Zincum Valerianicum, Zingiber Officinale, and combinations thereof.

Flowere ssences can also be included in cannabinoid compositions prepared according to the invention. Examples of suitable flower essences include, but are not limited to, Acacia, Actaea, Agrimony, Alpine Lily, Angel's Trumpet, AloeVera, Angelica, Basil, Apricot, Arnica Beech, Aspen, Avocado, Beech, Bee Balm, Black Cohosh, Baby Blue Eyes, Black-Eyed Susan, Blackberry, Bloodroot, Calendula, Bleeding Heart, California Fuchsia, California Pitcher Plant, Borage, Buttercup, California Wild Rose, California Poppy, CallaLily, Cerato, Canyon Dudleya, Chamomile, Cayenne, Cedar, Chaparral, Centaury, Centaurium erythraea or Centaurium umbellatum, Cerato, Cherry Plum, Chestnut Bud, Corn, Dandelion, Chicory, Cinquefoil, Coffee, Coreopsis, Crab Apple, Chrysanthemum, Clematis, Desert Dandelion, Deerbrush, Cosmos, Dill, Elm, Evening Primrose, Dogwood, Easter Lily, Eucalyptus, Fairy Lantern, Echinacea, Fawn Lily, Fig, Filaree, Gentian, Goldenrod, Forget-Me-Not, Golden Ear Drops, Golden Yarrow, Fuchsia, Garlic, Gorse, Honeysuckle, Heather, Hornbeam, Hibiscus, Hound's Tongue, Holly, Impatiens, Indian Paintbrush, Larch, Lily, Indian Pink, Larkspur, Iris, Iris douglasiana/Iris versicolor, Lady's Slipper, Cypripedium parviflorum/Cypripedium reginae, Lotus, Lavender, Love-Lies-Bleeding, Mariposa Lily, Madia, Magnolia, Milkweed, Mallow, Mimulus, Manzanita, Morning Glory, Motherwort, Mountain Pennyroyal, Mustard, Mountain Pride, Nasturtium, Mugwort, Nicotiana, Noni, Oak, Olive, Pine, Orange, Oregon Grape, Pansy, Passion Flower, Pear, Petunia, Pink Angel's Trumpet, Pink Monkeyflower, Penstemon, Pink Yarrow Achillea millefolium var. rubra, Peppermint, Poison Oak, Pomegranate, Queen Anne's Lace, Pretty Face, Quince, Purple Monkeyflower, Rabbitbrush, Quaking Grass, Red Chestnut, Red clover, Rescue Remedy, Rock Rose, Sacred Datura, Sagebrush, Scarlet Pimpernel, Rock Water Solarized spring water, Saguaro, Rosemary, Rose, Saint John's Wort, Scarlet Monkeyflower, Shasta Daisy, Scleranthus, Shooting Star, Scotch Broom, Snapdragon, Squash, Self-Heal, Star of Bethlehem, Star Thistle, Sweet Chestnut, Star Tulip, Strawberry, Sun Cup, Sweet Pea, Sticky Monkeyflower, Tansy, Sunflower, Thyme, Tiger Lily, Trillium, Violet, Walnut, Trumpet Vine, Vervain, Water Lily, Water Violet, Vine, White Chestnut, Wild Oat, Wild Rose, Yellow Star Tulip, Willow, Yerba Santa, Yarrow, Yucca, Zinnia, and combinations thereof

In one or more embodiments of the present disclosure the formulation comprising a cannabinoid and a tissue of the Capsicum fruit or an extract therefrom is an oral formulation for transmucosal delivery via the buccal route, labial route, gingival route or sublingual route or to be swallowed. Exemplary oral formulations include buccal gel, film, gum, chewable tablet or lozenge, orally disintegrating tablet, hard lozenge or lollipop, and liquids (e.g., solution or suspension). The oral formulations are prepared by mixing the active ingredients with one or more inactive ingredients, such as pharmaceutically acceptable excipients. In some cases, the one or more pharmaceutically acceptable excipients can be selected from sugar alcohols such as sorbitol, xylitol, maltitol, mannitol, isomalt, erythritol and combinations thereof, hydrocolloids, such as gelatin, gum acacia, carob gum, carrageenan, ghatti gum, guar gum, karaya gum, pectin, tragacanth gum, locust bean gum, xanthan gum and combinations thereof, and emulsifiers, surfactants, colorants, flavorants, taste modifiers, buffering agents, pH adjusting agents, stabilizers, and sweeteners.

In some embodiments the oral solution is packaged in order to facilitate the delivery of single doses, such as, for example, with a medicine dropper or in a spray/spritz-style package.

In some embodiments the oral formulation is a compressed tablet comprising chili pepper or a suitable extract therefrom along with at least one cannabinoid with a quantity of total cannabinoid of about 0.01 mg to 500 mg, along with at least one excipient to facilitate tablet compression. The content of chili pepper, or extract thereof can be varied based on the polyphenol, flavonoid, carotenoid, antioxidant/free radical scavenger, or capsaicinoid content of the tissue.

In some embodiments the oral formulation is a candy-style lozenge comprising chili pepper or suitable extract therefrom, along with at least one cannabinoid with a quantity of total cannabinoid of about 0.01 mg to 500 mg along with appropriate lozenge ingredients. The content of chili pepper, or extract thereof can be varied based on the polyphenol, flavonoid, carotenoid, antioxidant/free radical scavenger, or capsaicinoid content of the tissue. For example, in some cases, the amount of chili pepper or extract therefrom, varies based on capsaicin content to minimize unwanted pungency/heat.

In some embodiments the oral formulation is chewable. In some embodiments the oral formulation is a time released formulation. In some embodiments the oral formulation has been formulated to delay release of one or more of the active ingredients until the formulation has reached the stomach, small intestine, or large intestine respectively.

In some embodiments the active ingredients have been further formulated to facilitate parenteral delivery.

In some embodiments the formulations of the present invention are made by combining a formulated sugar alcohol intermediate to provide the at least one cannabinoid to the final formulation. The sugar alcohol formulated intermediate may be prepared by the procedure of the following Examples, which illustrate an embodiment of the present disclosure and should not be construed as to narrow its scope.

### EXAMPLES

### Sugar Alcohol Intermediate

i. Prepare a Concentrated Oil (CO) Formulate:

Calculate quantities of the one or more cannabinoid containing oils to add in order to achieve a desired potency/concentration and/or cannabinoid ratio. Heat oil, or oils, to workable fluidity (e.g., 50-60° C.).

For cannabinoid mixtures, add each cannabinoid containing oil into a clean appropriately sized vessel until reaching quantity setpoints determined by formulation calculations (±1 g desired mass). Seal vessel and heat, mixing well until homogeneous. Measure density of CO Formulate by pulling 1 mL in a pipette, taring the scale, and emptying pipette back into vessel as (average of pulls in triplicate). Confirm the concentration/ratio by quantitating the cannabinoids of interest in the CO Formulate via HPLC or other suitable analytical technique. Adjust formulation as needed to achieve target cannabinoids amount and/or ratio and verify adjusted formulation by HPLC or other suitable analytical technique.

ii. Prepare a Solvent Formulate:

Calculate quantities of CO Formulate and solvent (ethanol) needed. Heat CO Formulate to workable fluidity (50-60° C.). Slowly adding each component in an appropriately sized vessel until reaching setpoints determined by formulation calculations (1 g desired mass). Typical ratio is 1:1, i.e., 1 part CO Formulate to 1 part 200 proof ethanol, or similar solvent or mixture thereof. The Ethanol Formulated is sonicated in a bath sonicator.

In some cases, the Ethanol Formulate is homogenized with a probe sonicator. A taller, thinner vessel is preferred over a shorter, wider one for sonication. Sanitize probe by wiping with 200 proof ethanol before sonicating. Run sonicator per manufacturer's recommendations according to substrate volume, viscosity, composition, etc. Place vessel containing Ethanol Formulate in ice bath to keep solution cool during sonication. (e.g., dry ice and ethanol or water when available, or mixture of ice, water, and rock salt). Set temperature probe and sonicator probe in vessel. The tip of the sonicator probe should be about ⅓ from top of Ethanol Formulate. Ensure the sonicator probe never touches any glass or metal.

During the sonication process, the vessel is watched to verify it does not move or touch sonication probe. If temperature exceeds setpoint, the program should automatically pause. When this happens, wait for solution to reach below 30° C. and restart program. When finished, remove vessel from sonicator. Clean sonicator and temperature probes. Dispose of ice bath. The homogenized Ethanol Formulate is filtered (e.g., a 200 nm filter).

iii. Prepare a sugar alcohol (or sugar) Formulate:

Melt sugar or sugar alcohol. While isomalt is exemplified, other sugars or sugar alcohols can also be used. It takes about 55 minutes to melt 800 g isomalt in one 9″×13″ Pyrex baking dish. Larger quantities of isomalt can take several hours to melt completely. Using figures from Formulation calculations to provide a specific dose of cannabinoid (e.g., 10 mg/per dosage unit) measure isomalt into each Pyrex baking dish until total amount is achieved. Heat isomalt in oven until it becomes a uniform, clear liquid. Remove from oven immediately to avoid discoloration and off flavors (a burnt flavor).

While isomalt is heating, prepare mixing equipment and additional components (Quillaja extract (emulsifier) and gum Arabic (hydrocolloid)). A mixing container (such as a silicone pot or stainless pot) is cleaned and sanitized. When added to the isomalt, the quillaja extract will cause foaming. A suitably sized container will be at least 3 or 4 times the volume of the isomalt. Pre-measure quillaja extract per Formulation calculations in small beaker. Pre-measure gum Arabic in small beaker. When the isomalt is a clear, hot liquid, remove from oven and empty into silicone pot. While stirring, pour quillaja extract and half of the gum Arabic into isomalt. Keep stirring until foaming subsides and mixture is uniform in color.

Per Formulation calculations, add pre-determined amount of Ethanol Formulate. One method is to tare scale with vessel containing Ethanol Formulate. Pour Ethanol Formulate until scale shows the desired quantity has been removed. It is better to over-pour than to under-pour as the concentration can be reduced in the Final Formulation as needed. Ethanol will start evaporating once it hits hot isomalt, thereby removing the ethanol. This step should be done in a fume hood whenever possible. Keep stirring until color is uniform.

While hot, pour the molten mixture of isomalt, quillaja extract, gum Arabic, and cannabis oil mixture out onto suitable surface and spread, while hot, as thin of a layer as possible. Allow to cool to room temperature to solidify. The cooling process can be accelerated by cooling on and/or under dry ice.

iv. Prepare the Sugar Alcohol Intermediate

Once the isomalt formulate is completely cooled (no longer tacky and at room temperature) it can be broken into smaller pieces, aka “shards”. The hardness can be tested with a clean utensil—if the isomalt formulate does not allow deformation with a pointed utensil, it is ready to be broken. Cover the isomalt formulate to prevent isomalt shards from flying during the breaking process. Use hammer or hands or other suitable tools to break the solid sheet of isomalt formulate into small pieces.

Add the isomalt formulate shards to a collection vessel. Get a final weight of isomalt formulate added. Add remaining gum Arabic as a lubricant for size reduction. Total gum Arabic content is 1-2% of the mass of the isomalt formulate. Mix as well as possible to ensure the maximum amount of isomalt formulate is coated with gum Arabic powder. Record the amount of gum Arabic added. Mill Isomalt Formulate Shards into a powder of desired particle size using a mill or other appropriate equipment. The bulk powder was subjected to mechanical separation and filtering to provide a powder having a particle size of no greater than 600 microns.

### Exemplary Dosage Forms Containing the Sugar Alcohol Intermediate and Chili Pepper or Extract

The resulting water-soluble cannabinoid powder can be combined with at least one tissue from fruit of a Capsicum species (e.g., sweet or hot chili pepper) or extract therefrom and suitable tabletting excipients using conventional amounts, equipment, and methods for direct compression of a specific dosage of cannabinoid. Alternatively, the soluble cannabinoid powder can be combined with at least one extract or powder from a fruit of a Capsicum species, as active ingredients of a solution or suspension (e.g., an oral spray) with one or more inert ingredients, selected from the group consisting of organic solvents, emulsifiers, flavor modifiers, and antifoam agents, in oil or water. The solution or suspension can be dispensed from an atomizer or nebulizer, or other means for producing droplets of liquid.

In some embodiments of an oral formulation, the dosage form includes about 10 mg to 1000 mg per dose of the tissue, powder, or extract of the Capsicum species, such as about 100 mg to 500 mg per dose.

All formulations described are to be made in accordance with methods known to those having skill in the art and utilizing standard excipients, diluents, and processes.

While the present disclosure includes many embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

With respect to the above, it is to be understood that the invention is not limited in its application to the details of construction and to the arrangement of the components listed or the steps set forth in the description or illustrated in the drawings. The various apparatus and methods of the disclosed invention are capable of other embodiments, and of being practiced and carried out in various ways that would be readily known to those skilled in the art, given the present disclosure. Further, the terms and phrases used herein are for descriptive purposes and should not be construed as in any way limiting.

As such, those skilled in the art will appreciate that the conception upon which this disclosure is based may be utilized as a basis for designing other inventions with similar properties. It is important therefore that the embodiments, objects, and claims herein, be regarded as including such equivalent construction and methodology insofar as they do not depart from the spirit and scope of the present invention.

What is claimed is:

1. An oral formulation comprising:
at least one tissue from a fruit of a species of Capsicum, an extract therefrom, or a combination thereof;
at least one cannabinoid; and
a pharmaceutically acceptable excipient.

2. The oral formulation of claim 1, wherein the at least one tissue is dried and ground.

3. The oral formulation of claim 1, comprising about 10 mg to 1000 mg tissue.

4. The oral formulation of claim 3, comprising about 100 mg to 500 mg tissue.

5. The oral formulation of claim 1, comprising a quantity of tissue sufficient to achieve a predetermined amount of polyphenol, flavonoid, or capsaicin in the formulation.

6. The oral formulation of claim 1, wherein the at least one tissue or extract therefrom lacks capsaicin or substantially lacks capsaicin.

7. The oral formulation of claim 1, wherein the at least one tissue or extract therefrom is selected from a cultivar with a Scoville heat unit (SHU) value of less than 50,000.

8. The oral formulation of claim 1, wherein the formulation is an oral spray.

9. The oral formulation of claim 1, wherein the formulation is a compressed tablet.

10. The oral formulation of claim 1, wherein the formulation is a candy-style lozenge.

11. The oral formulation of claim 1, further containing at least one sugar alcohol.

12. The oral formulation of claim 1, further comprising a hydrocolloid selected from the group consisting of gelatin, gum acacia, carob gum, carrageenan, ghatti gum, guar gum, karaya gum, pectin, tragacanth gum, locust bean gum, xanthan gum and combinations thereof.

13. The oral formulation of claim 1, wherein the pharmaceutically acceptable excipient is selected from the group consisting of emulsifiers, surfactants, colorants, flavorants, taste modifiers, buffering agents, pH adjusting agents, stabilizers, sweeteners and combinations thereof.

14. The oral formulation of claim 1, wherein the at least one cannabinoid comprises a water-soluble powder including at least one cannabinoid oil, isomalt, a hydrocolloid, and an emulsifier.

15. The oral formulation of claim 14, wherein the hydrocolloid is gum acacia and the emulsifier is quillaja extract.

16. A method for improving health of a subject in need thereof comprising administering to the subject an oral formulation comprising:
at least one tissue from a fruit of a species of Capsicum, an extract therefrom, or a combination thereof;
at least one cannabinoid; and
a pharmaceutically acceptable excipient.

17. The method of claim 16, wherein the oral formulation is administered at least once a week for four weeks.

18. The method of claim 17, wherein the oral formulation is administered at least three times a week.

19. A method for reducing the probability of a cardiac event in a patient in need thereof comprising administering to the patient an oral formulation comprising:
at least one tissue from a fruit of a species of Capsicum, an extract therefrom, or a combination thereof;
at least one cannabinoid; and
a pharmaceutically acceptable excipient.
